(19) United States
(12) Patent Application Publication
(10) Pub. No.: US 2024/0238619 A1
(43) Pub. Date: Jul. 18, 2024

(54) NON-INVASIVE CELLULAR STIMULATION WITH UNIFORM ULTRASOUND FIELDS AND PREDICTION OF NEURONAL ACTIVITY RESULTING THEREFROM

(71) Applicants: The Regents of the University of California, Oakland, CA (US); The Salk Institute for Biological Studies, La Jolla, CA (US)

(72) Inventors: Aditya Vasan, San Diego, CA (US); James Friend, San Diego, CA (US); Jeremy Orosco, San Diego, CA (US); Sreekanth Chalasani, San Diego, CA (US); Uri Magaram, San Diego, CA (US)

(21) Appl. No.: 18/563,860

(22) PCT Filed: May 26, 2022
(86) PCT No.: PCT/US22/31218

§ 371 Date: Nov. 22, 2023

Related U.S. Application Data
(60) Provisional application No. 63/208,883, filed on Jun. 9, 2021.
(60) Provisional application No. 63/194,162, filed on May 27, 2021.
(60) Provisional application No. 63/194,168, filed on May 27, 2021.

(51) Int. Cl. A61N 7/00; B06B 1/06; G16B 5/00; G16H 20/30; G16H 40/67

(52) U.S. Cl. CPC A61N 7/00; B06B 1/0644; G16B 5/00; G16H 20/30; G16H 40/67; B06B 2201/76

(57) ABSTRACT

A system of ultrasound based cellular stimulation may include a stimulation apparatus and a stimulation controller. The stimulation apparatus may include a transducer element formed from a single crystal piezoelectric material such as lithium niobate. The stimulation apparatus may deliver high magnitudes of acoustic pressure relative to its dimensions (e.g., size, weight, and/or the like) and without significant heating. The stimulation controller may determine a cellular response to ultrasound stimulation such as membrane deflection and transmembrane voltage change. The stimulation controller may determine, based on the predicted cellular response, parameters for an ultrasound stimulation treatment for a patient such as a magnitude and/or duration of an ultrasonic stimulus for achieving a desired magnitude of membrane deflection and/or transmembrane voltage changes. The ultrasound stimulation treatment may be administered to the patient by the stimulation apparatus operating in accordance with the parameters.

### CROSS REFERENCE TO RELATED APPLICATIONS

This application claims priority to U.S. Provisional Application No. 63/194,168 entitled “SYSTEM TO PREDICT NEURONAL ACTIVITY DUE TO ULTRASOUND STIMULATION” and filed on May 27, 2021, U.S. Provisional Application No. 63/194,162, entitled “SYSTEM TO CREATE UNIFORM ULTRASOUND FIELDS IN ENCLOSED CAVITIES AND METHODS OF MANUFACTURE THEREOF” and filed on May 27, 2022, and U.S. Application No. 63/208,883, entitled “METHOD OF NON-INVASIVE CELLULAR STIMULATION” and filed on Jun. 9, 2021. The disclosures of the foregoing provisional applications are incorporated herein by reference in their entirety.

### TECHNICAL FIELD

This application relates generally to therapeutic applications of ultrasound and more specifically to ultrasound based cellular stimulation.

### BACKGROUND

Neurostimulation is a type of clinical therapy in which a subject's nervous system's activities are modulated using invasive or non-invasive means. Invasive means for stimulating neural activity include microelectrodes, which are inserted (or implanted) into the subject's brain and/or spinal cord (e.g., next to one or more neurons of interest) before an electric current is applied at a fixed frequency and time. Neural stimulation may also be achieved non-invasively using, for example, electromagnetic based mechanisms such as transcranial magnetic stimulation (TMS) and transcranial electric stimulation (TES). Neurostimulation can be used to treat a variety of neurological conditions and psychological disorders including, for example, paralysis, chronic pain, sensory loss, Alzheimer's disease, amyotrophic lateral sclerosis, persistent vegetative states, epilepsy, stroke related disability, tinnitus, multiple sclerosis, schizophrenia, traumatic brain injury, obsessive compulsive disorder (OCD), autism, substance abuse, addiction, and post-traumatic stress disorder (PTDS).

### SUMMARY

Systems, methods, and articles of manufacture, including computer program products, are provided for non-invasive ultrasound-based cellular stimulation. In one aspect, there is provided a system including a stimulation apparatus and a stimulation controller. The stimulation controller may include at least one data processor and at least one memory. The at least one memory may store instructions that cause operations when executed by the at least one data processor. The operations may include: predicting a cellular response to an application of ultrasound stimulation; determining, based at least on the predicted cellular response, one or more parameters of an ultrasound stimulation treatment for a patient; and administering, to the patient, the ultrasound stimulation treatment by at least causing the stimulation apparatus to operate in accordance with the one or more parameters.

In some variations, one or more features disclosed herein including the following features can optionally be included in any feasible combination. The stimulation apparatus may include a transducer element formed from a crystal piezoelectric material.

In some variations, the crystal piezoelectric material may be one or more of lithium niobate, lithium tantalate, quartz, and lithium tetraborate.

In some variations, a diffuser may be disposed on a first surface of the transducer element to reduce an intensity difference in an ultrasound stimulus generated by the transducer element.

In some variations, the diffuser may include one or more wells machined in a substrate to form a plurality of pillars having varying height.

In some variations, the plurality of pillars may be submillimeter in height.

In some variations, an epoxy backing may be disposed on a second surface of the transducer element.

In some variations, the stimulation apparatus may include a connector configured to provide an electrical connection between the transducer element and a power source.

In some variations, the connector may be a micro-miniature coaxial (MMCX) connector with rotary coaxial connections.

In some variations, the stimulation apparatus may further include a bracket and a mounting plate configured to house the connector and the transducer element.

In some variations, the bracket and the mounting plate may further house one or more magnets for securing the stimulation apparatus to a treatment area on the patient.

In some variations, the stimulation apparatus may be configured to deliver an acoustic pressure in response to a sinusoidal power input.

In some variations, a magnitude of the acoustic pressure relative to a dimension of the stimulation apparatus may be at least 1 MPa acoustic pressure per gram of the stimulation apparatus.

In some variations, the acoustic pressure may be in a range between 0.4 MPa to 0.6 MPa when the sinusoidal power input is in a range of 0.5 watts to 2 watts.

In some variations, the ultrasound stimulation treatment may include the stimulation apparatus delivering an ultrasonic stimulus to induce, in the patient, a cellular membrane deflection that causes a change in transmembrane voltage in multiple cell types.

In some variations, the one or more parameters may include a magnitude and/or a duration of the ultrasonic stimulus for achieving a desired magnitude of cellular membrane deflection and/or transmembrane voltage changes in the patient.

In some variations, the one or more parameters may include an amplitude, a frequency, and/or a peak pressure of the ultrasonic stimulus for achieving a desired magnitude of cellular membrane deflection and/or transmembrane voltage changes in the patient.

In some variations, the one or more parameters may be determined by applying a deflection model modelling the cellular response to the application of ultrasound stimulation.

In some variations, the deflection model may be generated based observations of cellular membrane deflection made using high-speed digital holographic microscopy (DHM) imaging.

In some variations, the deflection model may further model a change in transmembrane voltage based on a change in a capacitance of cellular membrane that corresponds to a change in an area of cellular membrane associated with the cellular membrane deflection.

In another aspect, there is provided a method for ultrasonic cellular stimulation. The method may include: predicting a cellular response to an application of ultrasound stimulation; determining, based at least on the predicted cellular response, one or more parameters of an ultrasound stimulation treatment for a patient; and administering, to the patient, the ultrasound stimulation treatment by at least causing a stimulation apparatus to operate in accordance with the one or more parameters.

In some variations, one or more features disclosed herein including the following features can optionally be included in any feasible combination. The stimulation apparatus may include a transducer element formed from a crystal piezoelectric material.

In some variations, the crystal piezoelectric material may be one or more of lithium niobate, lithium tantalate, quartz, and lithium tetraborate.

In some variations, a diffuser may be disposed on a first surface of the transducer element to reduce an intensity difference in an ultrasound stimulus generated by the transducer element.

In some variations, the diffuser may include one or more wells machined in a substrate to form a plurality of pillars having varying height.

In some variations, the plurality of pillars may be submillimeter in height.

In some variations, an epoxy backing may be disposed on a second surface of the transducer element.

In some variations, the stimulation apparatus may include a connector configured to provide an electrical connection between the transducer element and a power source.

In some variations, the connector may be a micro-miniature coaxial (MMCX) connector with rotary coaxial connections.

In some variations, the stimulation apparatus may further include a bracket and a mounting plate configured to house the connector and the transducer element.

In some variations, the bracket and the mounting plate may further house one or more magnets for securing the stimulation apparatus to a treatment area on the patient.

In some variations, the stimulation apparatus may be configured to deliver an acoustic pressure in response to a sinusoidal power input.

In some variations, a magnitude of the acoustic pressure relative to a dimension of the stimulation apparatus may be at least 1 MPa acoustic pressure per gram of the stimulation apparatus.

In some variations, the acoustic pressure may be in a range between 0.4 MPa to 0.6 MPa when the sinusoidal power input is in a range of 0.5 watts to 2 watts.

In some variations, the ultrasound stimulation treatment may include the stimulation apparatus delivering an ultrasonic stimulus to induce, in the patient, a cellular membrane deflection that causes a change in transmembrane voltage in multiple cell types.

In some variations, the one or more parameters may include a magnitude and/or a duration of the ultrasonic stimulus for achieving a desired magnitude of cellular membrane deflection and/or transmembrane voltage changes in the patient.

In some variations, the one or more parameters may include an amplitude, a frequency, and/or a peak pressure of the ultrasonic stimulus for achieving a desired magnitude of cellular membrane deflection and/or transmembrane voltage changes in the patient.

In some variations, the one or more parameters may be determined by applying a deflection model modelling the cellular response to the application of ultrasound stimulation.

In some variations, the deflection model may be generated based observations of cellular membrane deflection made using high-speed digital holographic microscopy (DHM) imaging.

In some variations, the deflection model may further model a change in transmembrane voltage based on a change in a capacitance of cellular membrane that corresponds to a change in an area of cellular membrane associated with the cellular membrane deflection.

In another aspect, there is provided a computer program product including a non-transitory computer readable medium storing instructions. The instructions may cause operations may executed by at least one data processor. The operations may include: predicting a cellular response to an application of ultrasound stimulation; determining, based at least on the predicted cellular response, one or more parameters of an ultrasound stimulation treatment for a patient; and administering, to the patient, the ultrasound stimulation treatment by at least causing a stimulation apparatus to operate in accordance with the one or more parameters.

In another aspect, there is provided an apparatus for ultrasonic cellular stimulation. The apparatus may include: means for predicting a cellular response to an application of ultrasound stimulation; means for determining, based at least on the predicted cellular response, one or more parameters of an ultrasound stimulation treatment for a patient; and means for administering, to the patient, the ultrasound stimulation treatment by at least causing a stimulation apparatus to operate in accordance with the one or more parameters.

In another aspect, there is provided a stimulation apparatus that includes: a transducer element formed from a crystal piezoelectric material; a diffuser disposed on a first surface of the transducer element to reduce an intensity difference in an ultrasound stimulus generated by the stimulation apparatus; an epoxy backing disposed on a second surface of the transducer element; a connector configured to provide an electrical connection between the transducer element and a power source; and a bracket and a mounting plate configured to house the connector and the transducer element.

In some variations, one or more features disclosed herein including the following features can optionally be included in any feasible combination. The stimulation apparatus may be coupled with a stimulation controller configured to predict a cellular response to an application of ultrasound stimulation, determine, based at least on the predicted cellular response, one or more parameters of an ultrasound stimulation treatment for a patient, and administer, to the patient, the ultrasound stimulation treatment by at least causing the stimulation apparatus to operate in accordance with the one or more parameters.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to ultrasound based cellular stimulation, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of an ultrasound-based cellular stimulation system, in accordance with some example embodiments;

FIG. 2A depicts an exploded perspective view of an example of an ultrasound-based cellular stimulation apparatus, in accordance with some example embodiments;

FIG. 2B depicts an assembled perspective view of an example of an ultrasound-based cellular stimulation apparatus, in accordance with some example embodiments;

FIG. 2C depicts a perspective view of an example of a mounting plate for an ultrasound-based cellular stimulation apparatus, in accordance with some example embodiments;

FIG. 2D depicts a perspective view of an example of a bracket for an ultrasound-based cellular stimulation apparatus, in accordance with some example embodiments;

FIG. 3 depicts the magnitude of pressure delivered by an example of an ultrasound-based cellular stimulation apparatus, in accordance with some example embodiments;

FIG. 4 depicts an experimental setup with an example of an ultrasound-based cellular stimulation apparatus, in accordance with some example embodiments;

FIG. 5 depicts another experimental setup with an example of an ultrasound-based cellular stimulation apparatus, in accordance with some example embodiments;

FIG. 6 depicts a) a diffuser design based on Schröder's method of quadratic-residue sequences to determine well depth. The wells were machined in glass using a KrF excimer laser system with a custom metal mask to restrict beam width. The machined depth of the pillars is up to 309 μm. b) The glass diffuser block was then c) bonded to a transducer operating in the thickness mode at 7 megahertz using an ultraviolet light-curable epoxy. (c) A scanning laser Doppler vibrometer image of the diffuser face in the time domain shows phase differences corresponding to pillar heights (normalized autocorrelation >0.73);

FIG. 7 depicts 2D instantaneous pressure profile for the a) domain b) without and c) with the diffuser. Human embryonic kidney (HEK) cells were placed in the middle of the (light blue) fluid domain with an objective lens for an inverted microscope at top. The pressure field was generated by defining a sinusoidal pressure displacement to the transducer face, located at the bottom of the domain. Pressure maps were extracted from the results in 1 us time steps over grid points specified within the domain. A 2D autocorrelation was performed on this grid over time; each X, Y point plotted from the d,e) results of the autocorrelation corresponds to a single point in the (a) domain. Spatial and temporal periodicity was observed through the existence of a large value of autocorrelation over the domain (d) without the diffuser. e) With the diffuser, however, the autocorrelation was much smaller for most of the domain;

FIG. 8 depicts calculated isofrequency contour at the driving frequency a) without and b) with the diffuser. The circular profile traced by both cases corresponds to the wave vector in water at the driving frequency. (a) Without the diffuser, most of the wave is isolated to propagation along the Y axis. Using (b) a diffuser with the transducer produced wave vectors spread around this circular profile, indicating a more uniform distribution of the ultrasound. Calculating c) the normalized root mean square (RMS) pressure 10 μm above the coverslip (inset; halfway between the transducer and objective lens) indicates a smaller difference between the minimum and maximum RMS values (red) with the diffuser than (blue) without it. The diffuser produces a much more uniform pressure distribution across the coverslip plane;

FIG. 9 depicts a) the experimental setup for confirming the utility of the diffuser in an in vitro setting consists of an upright epi-fluorescent microscope, an immersion objective, and a chamber that houses cells on a coverslip and the diffuser assembly. Standing wave components may exist between the transducer and the coverslip and between the coverslip and the immersion objective. The calcium concentration before and after ultrasound stimulation in the same field of view is b) shown for HEK cells expressing hsTRPA1. Comparison of fluorescence changes as measured using GCaMP6f reporters with respect to time for two cases, c) without (control) and with the diffuser, shows an increase in both number and magnitude of cells being activated upon introduction of the diffuser. d) HEK cells expressing TRPAI show a greater response to ultrasound stimuli with a diffuser present in comparison to both no diffuser and dTom-based (red fluorescent protein) controls. The magnitude of the response when the diffuser is used is significantly greater (over twice as high) than when the diffuser is not used (n \grave{1}/4 76 221, p<0.0001 by a Mann-Whitney test);

FIG. 10 depicts pressure being measured using a) a fiber optic hydrophone at two different locations along the anterior-posterior axis: the ventral surface of the pons (triangle) and the ventral surface of the anterior olfactory bulb (circle). The measured pressure is b) uniform across different brain regions for different input powers above 0.2 MPa (minimum detectable pressure using our setup), indicating that the diffuser creates a uniform acoustic field within the skull cavity. This eliminates the influence of the cranial structure and ensures that only the brain regions that have been infected with hsTRPA1 will be sensitive to ultrasound stimuli. In comparison, the control case without the diffuser shows a threefold deviation in pressure values for the same input power for different brain regions along the anterior posterior axis;

FIG. 11 an example of high-speed digital holographic microscopy (DHM) imaging of membrane deflection. The deflection of the membrane under the influence of ultrasound was visualized using a) high-speed digital holographic microscopy (DHM). The digital holographic microscopy setup included a lithium niobate transducer driven by a signal generator and an amplifier at 6.72 megahertz. The cells were mounted on a coverslip and placed in a custom perfusion chamber maintained at 37° C. Digital holographic microscopy enables the b) quantitative reconstruction of phase images acquired by the high-speed camera at 40 000 frames s−1. Each recording began with 25 miiliseconds of no stimulus as a baseline, followed by a 50-millisecond ultrasound stimulus, and ended with a 25 milliseconds baseline. c) The maximum deflection from the mean position was found to be 100-400 nanometers, with a median deflection of 214 nm for HEK293 cells and 160 nm for neurons (N=30 for each cell type). Reconstructed phase profiles are shown for different cell types: d) HEK293 cells, e) neurons, and f) neuronal clusters. Displacement was measured as a function of distance along the green lines provided in the (d-f) contour plots and were g-i) plotted with (red line plot, max displacement during stimulus) and without (green plot, Baseline) ultrasound stimulus. A distance of “zero” in (g-i) is at the left end of the green line in (d) and (e) and at the bottom of the green line in (f). For the (green) baseline displacement, note the mean and 95% confidence intervals are provided. The maximum variation throughout all baseline responses was less than +20 nanometers;

FIG. 12 depicts an example of prediction of membrane deflection due to ultrasound. Ultrasound results in a) membrane deflection that triggers a transmembrane electrical response. The cell membrane bilayer stretches, increasing its area, and the outer leaflet of the bilayer will likely deflect more than the inner leaflet due to the presence of cytoskeletal components such as actin and microtubules that anchor the inner leaflet. Two of the factors that affect membrane displacement are surface tension of the lipid membrane and the length under consideration. The model b) predicts displacements between 100-400 nanoseconds for dimensions that correspond to the size of a cell (5-20 μm) and is within the limits observed using the digital holographic microscopy. The response is c) dynamic, with snapshots of the predicted deflection at different times (in ms) across a 10 μm wide membrane section that is anchored at the ends. The maximum deflection occurs when the stimulus is first provided and there is a balance between viscous dissipation and conservative effects of inertia and surface tension which lead to sustained wavemodes on the membrane at the millisecond timescale (observed response). A low-pass temporal filter of the membrane's center displacement at 5 μm indicates d) an oscillatory deflection over the stimulus duration of 5 milliseconds;

FIG. 13 depicts the displacement driven capacitance changes that result in action potential generation. a-e) Simulations help inform the development of stimulus parameters, in terms of time and pressure amplitude; note that throughout (a-e) 0.5 MPa stimulation is red while 1 MPa is blue. The capacitance changes are plotted over the stimulus duration (5 ms) for a) 0.5 and b) 1 MPa with the corresponding area changes that cause c) capacitance fluctuations. The capacitance fluctuations produce depolarization at 1 MPa, but not at 0.5 MPa, indicating d) the presence of a pressure threshold to stimulate neurons. e) Over a longer 50 ms stimulus, the action potential evolves quite differently over time for the two acoustic pressures. At lower pressures, longer stimuli may be necessary to produce action potentials. f) In vitro current clamp electrophysiology was used to verify the predictions of the model and shows that the presence of a preliminary spike followed by oscillations in voltage across the membrane;

FIG. 14 depicts a flowchart illustrating an example of a process for ultrasound based cellular stimulation, in accordance with some example embodiments; and

FIG. 15 depicts a block diagram illustrating an example of a computing system, in accordance with some example embodiments.

When practical, similar reference numbers denote similar structures, features, or elements.

### DETAILED DESCRIPTION

Neurostimulation can be used to treat a variety of neurological conditions and psychological disorders including, for example, paralysis, chronic pain, sensory loss, Alzheimer's disease, amyotrophic lateral sclerosis, persistent vegetative states, epilepsy, stroke related disability, tinnitus, multiple sclerosis, schizophrenia, traumatic brain injury, obsessive compulsive disorder (OCD), autism, substance abuse, addiction, and post-traumatic stress disorder (PTDS). When applied to certain organs, neurostimulation can modulate organ functions. For example, when applied to the pancreas, neurostimulation can regulate the secretion of digestive enzymes and hormones such as insulin, glucagon, pancreatic polypeptide, and somatostatin.

Nevertheless, existing methods to stimulate neural activity include electrical, optical, and chemical techniques. They have enabled the development of novel therapies that are used in clinical settings, in addition to helping understand aspects of neural function and disease mechanisms. Despite their beneficial impact, these approaches are fundamentally limited. Electrical stimulation is invasive, requiring direct contact with the target of interest. Inserting electrodes into the brain may lead to inflammation, bleeding, cell death, and local cytokine concentration increases in microglia that precipitate astrocyte formation around the electrodes that, in turn, reduce long-term effectiveness. In addition, it may have nonspecific effects depending on the electric field generated by the electrodes and the stimulation parameters used. Transcranial direct current stimulation and transcranial magnetic stimulation are new and non-invasive, yet they have poor spatial resolution on the order of 1 centimeter. Furthermore, approaches combining genetic tools with light or small molecules achieve cellular specificity. Optogenetics, which involves the use of light and genetically encoded membrane proteins, has enabled elucidation of cellular circuits in animal models. However, it remains an invasive technique and applications are limited by the depth of penetration of light in tissue. By contrast, chemogenetics, using small molecule sensitive designer receptors, is limited by poor temporal resolution and is unfortunately impractical for many neural applications that require millisecond response times.

Ultrasound can overcome the limitations of these methods. It is non-invasive and exhibits a high spatiotemporal resolution (<1 millimeter and 1 millisecond) in comparison to existing techniques. Improvements in the spatial resolution through transfection of mechanosensitive proteins currently come at the cost of a minimally-invasive procedure to directly inject the vector into the target tissue, though there may soon be non-invasive alternatives. The spatial resolution of ultrasound is governed by the wavelength of operation and is about 1.5 millimeter at 1 megahertz in tissue. The temporal resolution is dependent on the pulse duration of stimulation and may be as short as a single time period T, T=1/f where f is the operating frequency. The frequency choice is dictated by the depth and size of the target region in traditional focused ultrasound neuromodulation. Accordingly, in some example embodiments, a ultrasonic stimulation apparatus may be used to induce cellular stimulation. Various examples the ultrasonic stimulation apparatus disclosed herein may be capable of driven at a high power to deliver high magnitudes of acoustic pressure relative to its dimensions (e.g., size, weight, and/or the like) and without significant heating. Moreover, various examples of the ultrasonic stimulation apparatus disclosed herein may include mechanisms, such as a diffuser, to reduce interference between radiated and reflected ultrasound, produce diffuse and uniform ultrasound throughout a treatment region (e.g., an enclosed cavity and/or the like), and transduce sufficient power to produce adequate acoustic pressure (e.g., 0.4 MPa) in the treated tissue, all while remaining sufficiently small and lightweight.

Despite recent experimental and clinical developments, and progress in exploring the sonogenetic and ultrasonic-to-chemical action mechanisms, there is no convincing, overarching explanation for the observations reported in vitro or in vivo. Some of the proposed mechanisms include cavitation, indirect auditory signaling in vivo and increased lipid clustering resulting in a change in the membrane tension. These studies have either been conducted on time scales that are orders of magnitude larger than those used for ultrasound neuromodulation, lack robust imaging techniques that operate at timescales relevant to the frequency of stimulation, or use incorrect stimulation thresholds that are orders of magnitude lower than values reported in experimental work. Additionally, studies often treat surface tension, membrane composition, and membrane stresses as a single term, membrane fluidity. This term lacks rigorous physical description and is assigned a value based on relative fluorescence intensity changes. The imprecision of this description makes it difficult to isolate the influence of the measurable physical mechanisms of which it is comprised. A model using membrane fluidity leaves the explanation of the biophysical phenomenon incomplete.

More broadly, action potentials are known to appear in phase with the cell membrane's deflection. A key limitation in validating existing models is the inability to measure physical motion across the vast differences in spatiotemporal scales. The ultrasound signal is on the order of 1 megahertz and is three orders of magnitude faster than the electrical response of a cell. The wavelength of ultrasound in tissue at these frequencies is orders of magnitude larger than the membrane thickness. Existing methods to measure cell membrane deflection include contact-based atomic force microscopy, which has high spatial resolution but poor temporal resolution and lacks the ability to simultaneously scan multiple points. Optical tweezers have been used for over twenty years, but only produce results from slow to static deformation of cells and often require attachment of beads or other structures that reduce the measurement to just a few spatial points. Traditional digital holographic imaging is slow but offers high spatial resolution across a large field of view.

In some example embodiments, high-speed digital holographic microscopy (DHM), which provides higher resolution in both space and time than previous methods, may be deployed to analyze the dynamics of the cell membrane due to ultrasound. Application of high-speed digital holographic microscopy (DHM) provides the first three dimensional (3D) visualization of cell membrane deflection due to an ultrasound stimulus. Current clamp electrophysiology may be used in an environment of intense ultrasound to monitor ultrasound-driven, real-time changes in voltage across the membrane in single neurons in vitro. Furthermore, neuronal depolarization driven by membrane deflection from applied ultrasound stimulus may be predicted by various examples of a biophysical analytical model disclosed herein. Experimental results confirm the predictions made by the biophysical model, both with regard to membrane deflection and voltage changes. It should be appreciated that these findings provide insight into the effects of ultrasound on cells and cell signaling, the understanding of which is vital tosonogenetics and its clinical application.

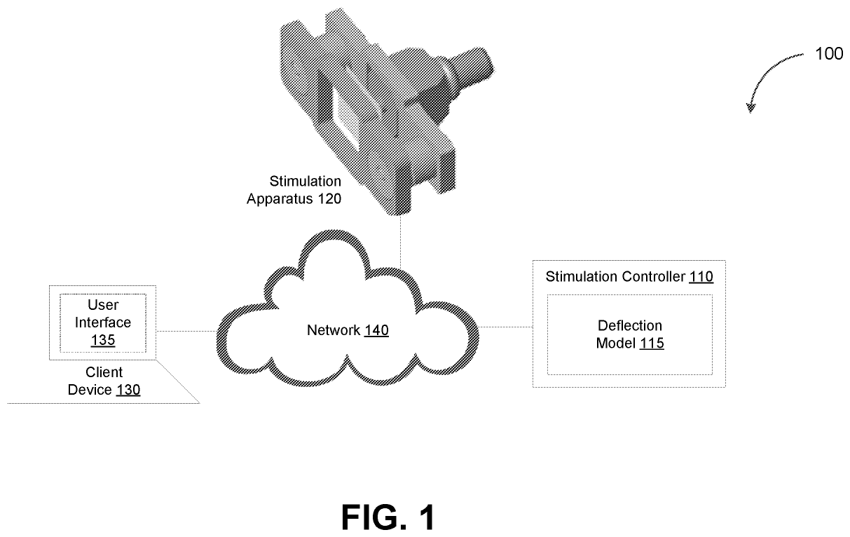

FIG. 1 depicts a system diagram illustrating an example of an ultrasound-based cellular stimulation system 100, in accordance with some example embodiments. Referring to FIG. 1, the ultrasound-based cellular stimulation system 100 may include a stimulation controller 110, a stimulation apparatus 120, and a client device 130. In the example of the ultrasound-based cellular stimulation system 100 shown in FIG. 1, the stimulation controller 110, the stimulation apparatus 120, and the client device 130 may be communicatively coupled via a network 140. The client device 130 may be a processor-based device including, for example, a mobile device, a wearable apparatus, a personal computer, a workstation, an Internet-of-Things (IOT) appliance, and/or the like. The network 140 may be a wired network and/or wireless network including, for example, a public land mobile network (PLMN), a local area network (LAN), a virtual local area network (VLAN), a wide area network (WAN), the Internet, and/or the like.

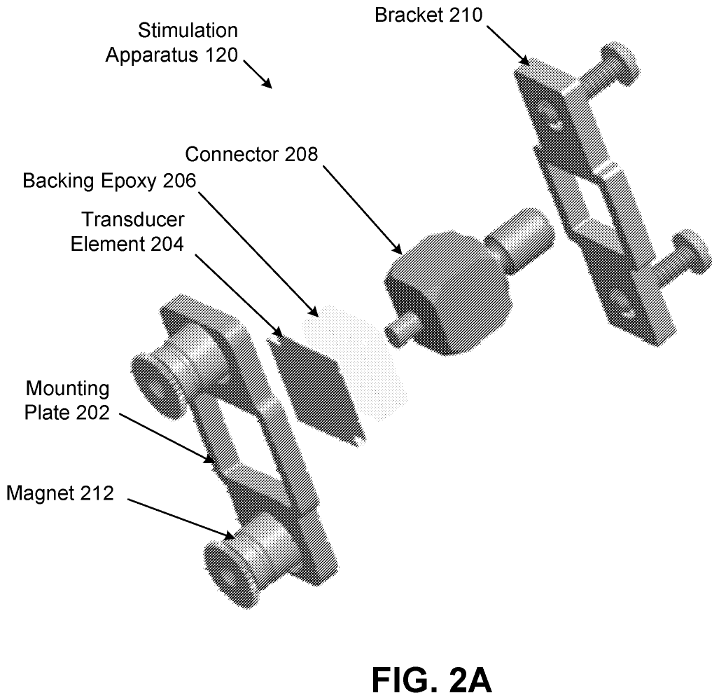

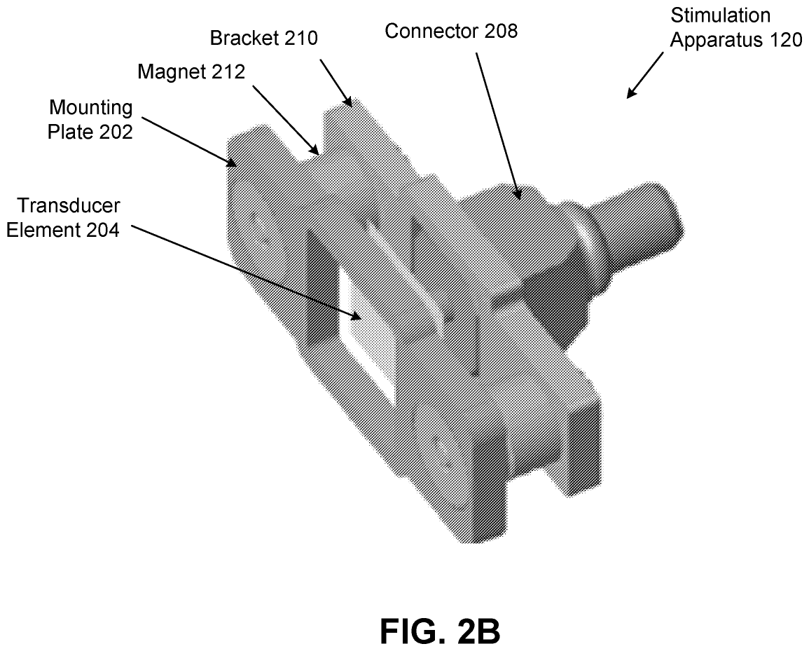

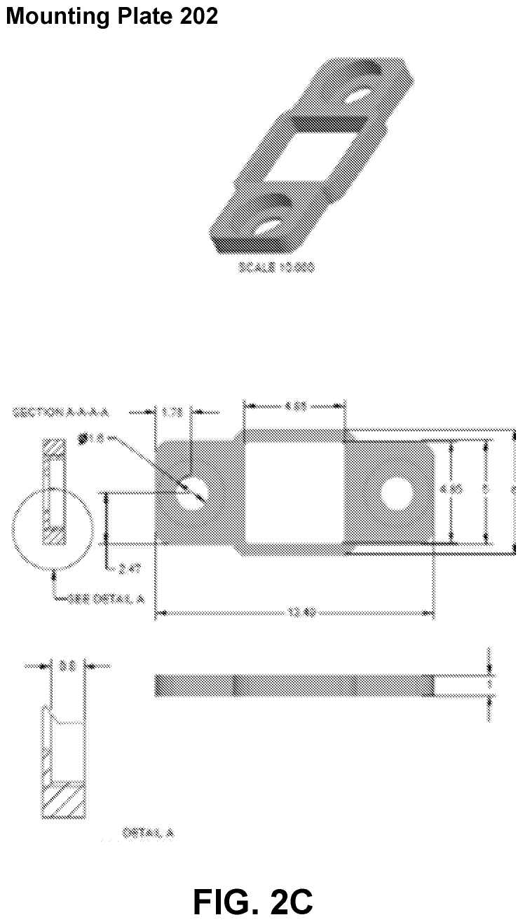

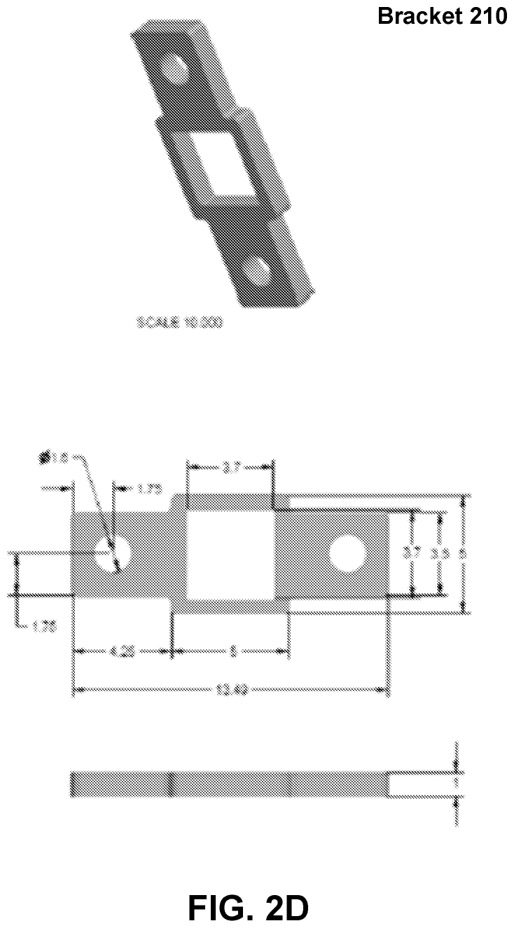

FIGS. 2A-B depict an example of the stimulation apparatus 120, in accordance with some example embodiments. According to some example embodiments, the stimulation apparatus 120 may be an ultrasound-based cellular stimulation apparatus configured to generate high-frequency sound waves to perturb cell membranes and stimulate ionic influx (e.g., calcium and/or the like) into the cell. It should be appreciated that various examples of the stimulation apparatus 120 disclosed herein may be adapted for performing various therapies such as non-invasive neuromodulation on live subjects such as animals. Referring to FIGS. 2A-B, the stimulation apparatus 120 may include a connector 208 (e.g., a micro-miniature coaxial (MMCX) connector with rotary coaxial connections. The stimulation apparatus 120 may also include a transducer element 204 (e.g., lithium niobate (127.86 YX cut) with titanium and gold coating) with a backing epoxy 206 (e.g., a two-part epoxy). The stimulation apparatus 120 may further include a mounting plate 202 and a bracket 210 configured to house one or more magnets 212 (e.g., neodymium magnets) for securing the stimulation apparatus 120 to a subject. The stimulation apparatus 120 may include solder for various electrical connections.

In some example embodiments, the transducer element 204 of the stimulation apparatus 120 may include a single crystal piezoelectric material (e.g., lithium niobate, lithium tantalate, quartz, lithium tetraborate, and the like) with a certain fundamental frequency (e.g., such as approximately 7 megahertz). The piezoelectric material can be driven at high powers without significant heating. As shown in FIGS. 2A-B, the transducer element 204 (e.g., the piezoelectric material) may be housed in an enclosure, such as the connector 208 that enables interfacing with coaxial connectors. Acoustic waves generated by the transducer element 204 may propagate through tissue and cause membrane perturbation. Capacitive changes occur across the membrane, resulting in cellular activity.

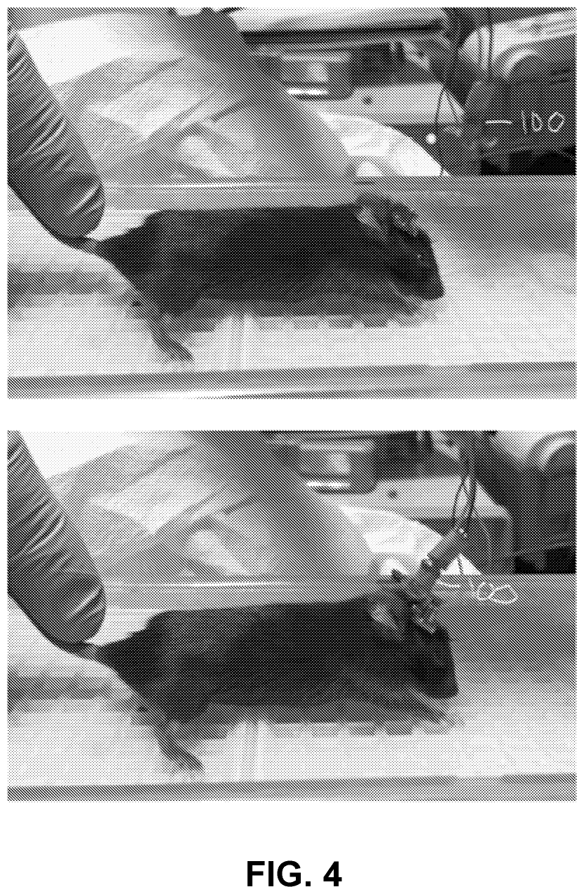

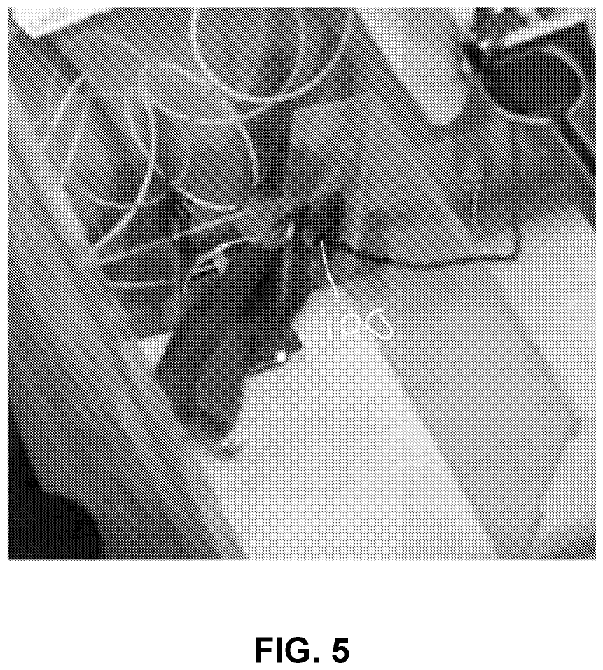

The stimulation apparatus 120 may be controlled by the stimulation controller 110, which may include a signal generator and amplifier, such that parameters can be varied in order to result in stimulation, (e.g., stimulation time, duty cycle, and pressure). For example, the stimulation apparatus 120 may include one or more magnets 212 (e.g., two 1-millimeter diameter neodymium magnets) that enable the stimulation apparatus 120 to be secured to the mounting plate 202 with similar magnets of opposing polarity. The stimulation apparatus 120 may be coupled to tissue using ultrasound gel or a water reservoir in order to enable efficient acoustic wave transmission between the stimulation apparatus 120 and tissue of the subject. According to some example embodiments, the stimulation apparatus 120 may weigh less than a gram, so that it could be mounted on a freely moving subject (e.g., a mouse) and output pressures of up to 2 MPa. FIGS. 4-5 depict experimental setups where an example of the stimulation apparatus 120 is coupled to a live subject (e.g., a mouse). The low weight, high pressure output, and easy attachment mechanism coupled with a scalable manufacturing process make examples of the stimulation apparatus 120 disclosed herein superior in comparison to existing devices.

Systems to modulate cellular activity typically involve using electrical, optical, or chemical systems. As noted, the limitation with these approaches is that they are either invasive or have poor temporal resolution. Contrastingly, the stimulation apparatus 120 provides a non-invasive means for neuromodulation. Notably, examples of the stimulation apparatus 120 disclosed herein are configured to overcome various limitations associated with size, power consumption, temperature rise, and biocompatibility. For example, as noted, the stimulation apparatus 120 may be implemented to have a low weight (e.g., less than a gram) while being capable of being mounted on a live subject (e.g., using a magnetic attachment mechanism). The low weight and small dimensions of the stimulation apparatus 120, particularly relative to its power output, renders the stimulation apparatus 120 suitable for a variety of applications regardless of the size of the subject. The transducer element 204 of the stimulation apparatus 120 may include a crystal piezoelectric element (e.g., crystalline lithium niobate, lithium tantalate, quartz, and lithium tetraborate, and/or the like) capable of being driven at high powers without significant losses. The response of cellular populations to ultrasound stimuli is dependent on pressure generated by the transducer. Thus, using a single crystal piezoelectric element ensures efficient conversion of electrical energy to mechanical energy through the converse piezoelectric effect with minimal hysteresis. This is an advantage over conventional materials used in transducers, such as lead zirconate titanate (PZT), where the losses are far more significant. Conventional transducer materials also contains toxic materials, such as lead, in the grain boundaries, and are thus non-biocompatible.

In some example embodiments, the stimulation apparatus 120 may be manufactured in an efficient and practical manner. For example, the manufacturing process starts by making modifications to the connector 208 (e.g., the micro-miniature coaxial (MMCX) connector) to accommodate the transducer element 204, the backing epoxy 206, and various electrical connections. The transducer element 204 may be prepared separately using clean room fabrication processes and compositions. Upon modification, the connector 208 may be coated with a low volume of solder and contact is established with one face of the transducer element 204 before soldering to ensure a permanent bond. The region between the transducer element 204 and the connector 208 may be covered with shrink wrap and a pipette with microliter adjustments is used to dispense material forming the backing epoxy 206 between the bottom face of the transducer element 204 and the connector 208. This not only effects the acoustic properties of the stimulation apparatus 120 but also ensures that the stimulation apparatus 120 remains structurally intact. The backing epoxy 206 may be kept minimal in order to prevent the backing epoxy 206, which can generate heat when subjected to the power applied to the transducer element 204, from becoming a source of excessive heat. A 36-gauge wire is stripped and a solder joint is established between the top face of the transducer element 204 and the stimulation apparatus 120. The stimulation apparatus 120 may be covered with shrink wrap again and a microliter pipette is used to dispense epoxy over the 36 gauge wire. The rotary joint of the connector 208 may be modified to reduce interference between mating connectors. The mounting plate 202 and the bracket 210, which may be rendered in stainless steel, are then assembled and attached permanently to the stimulation apparatus 120 using the same epoxy. Finally, the one or more magnets 212 (e.g., the pair of neodymium magnets) are bonded to the mounting plate 202 and the bracket 210.

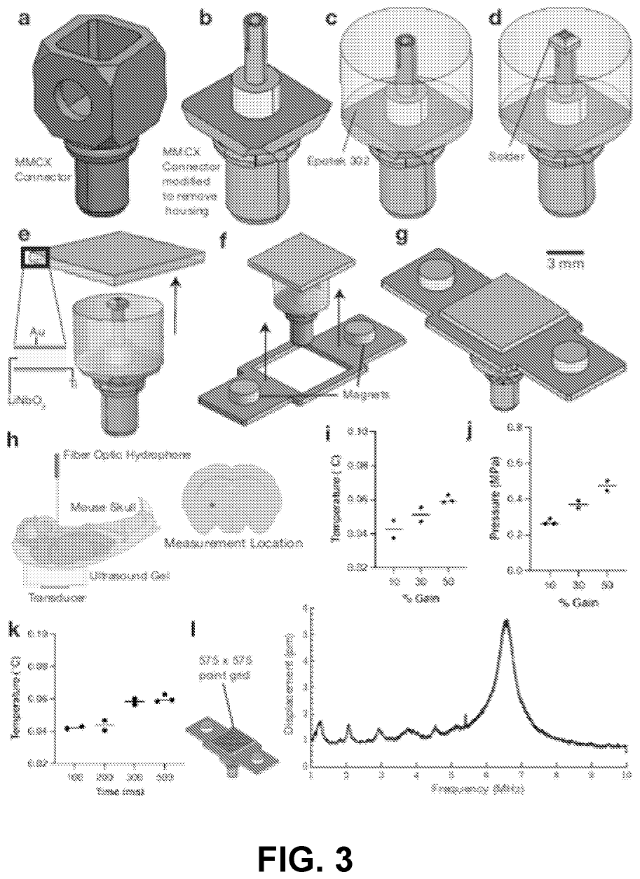

FIG. 3 depicts an miniaturized version of the stimulation apparatus 120 for use with a freely moving small scale subject such as a mouse. Conventional ultrasound transducers are typically made for imaging and can result in heating or tissue damage when used for cellular stimulation. As noted, in some example embodiments, the transducer element 204 of the stimulation apparatus 120 may be formed from a single crystal piezoelectric material, such as crystalline lithium niobate, using the manufacturing process illustrated in FIG. 3(a) to (g). Configured as such, the stimulation apparatus 120 is capable of delivering sufficient power, such as through the skull of a subject, without triggering any significant temperature changes. The stimulation apparatus 120 may be snapped onto the mounting plate 202 mounted on the subject, for example, at the treatment area, using the one or more magnets 212 (e.g., neodymium magnets) and the entire assembly for the stimulation apparatus 120 including the mounting plate 202 may weigh less than a gram. The pressure and temperature change generated by the stimulation apparatus 120 were measured using a fiber optic hydrophone in the striatum in the manner shown in FIG. 3(h). As shown in FIGS. 3(i) and (k), the stimulation apparatus 120 is capable of triggering significant pressure changes for the various input powers shown in FIG. 3(j) without causing insignificant temperature changes. The vibration amplitude was characterized using a laser doppler vibrometry scan of the face of the transducer element 204. FIG. 3(i) shows the presence of a narrow band peak centered at 6.5 megahertz.

Ultrasonic stimulation can be used in a variety of neurological applications for imaging tissue, disrupting blood-brain barriers, invasive and non-invasive neuromodulation, and thrombolysis. In these cases, ultrasound is typically focused at a certain depth defined by a phased array of transducers or an acoustic lens formed by a concave surface at the exit face of the transducer. A fundamental limitation of these approaches is the formation of standing waves due to resonant reflections within the skull cavity formed by the relatively high impedance of the skull's cortical bone compared to the tissue of the brain, and thus regions of either extremely high intensity or zero intensity at every one-half of an acoustic wavelength. The presence of these local maxima may lead to unintended bioeffects in tissues when applied to neuromodulation, including heating or even tissue damage from cavitation. Such adverse effects in tissue have been reported during ultrasound-driven thrombolysis and blood-brain barrier disruption. Additionally, commonly used transducer materials such as lead zirconate titanate (PZT) also have limitations in high power applications at frequencies above 1 megahertz, producing losses, hysteresis, and internal (ohmic) heating as current passes through elemental lead present at the morphological grain boundary, which limits the use of broadband stochastic signal generation for reducing the impact of standing wave generation. To overcome these limitations, the stimulation apparatus 120 may be implemented using loss-free, single-crystal piezoelectric material to generate single-frequency ultrasound output in the 1-20 megahertz range with an attached diffuser. Implemented as such, the stimulation apparatus 120 may be capable of delivering a spatiotemporally diffuse ultrasound field for various applications, including sonogenetics.

Sonogenetics relies on genetically engineering cells to be more sensitive to mechanical stimuli using membrane bound proteins. This technique eliminates the need for focused ultrasound by ensuring that targeted neural circuits are the only ones that will respond to an ultrasound stimulus. Currently, the method of transfection is an invasive method that uses adeno-associated virus (AAV) delivered by syringe. One protein in particular, human transient receptor potential A1 (hsTRPA1), produces ultrasound-evoked responses in several cell types. This response is due to deformation and consequent stretching of the cell membrane from exposure to ultrasound that, in turn, leads to a change in the membrane capacitance between a chemically induced potential difference from inside to outside the cell. This produces a current sufficient to cause hsTRPA1 responses. One limitation of sonogenetics is that existing transducers producing planar or focused ultrasound, typically at a single frequency, are unsuitable. Furthermore, in many applications, the transducer must be small to avoid affecting animal behaviour, which excludes phased array based approaches. Transducers that can be attached to a freely moving subject enable the study of neural circuits in their native state, without the confounding effects of anaesthesia as reported in past studies. However, no small broadband transducers exist that might facilitate the generation of spatiotemporally random ultrasound noise from a similarly random input signal at sufficient power for sonogenetics. Moreover, commonly used animal models like rodents have small heads with a typical mass of 3-4 grams, less than half the mass of all commercially available or research-based power ultrasound transducers.

The effective implementation of sonogenetics requires a very different transducer design. It must reduce interference between the radiated and reflected ultrasound, produce diffuse and uniform ultrasound throughout the region, and transduce sufficient power to produce over 0.4 MPa acoustic pressure in tissue, all while remaining sufficiently small and light enough to attach to the head of a live, freely moving subject (e.g., a mouse). This would enable the study of neural circuits in the subject's native state. In addition, these devices also have to avoid generating electromagnetic signals and localized temperature changes. If left to appear, electromagnetic and thermal phenomena may conflate with the effects of ultrasound on the cells in sonogenetics experiments, reducing one's confidence in ultrasound's contribution to the observations.

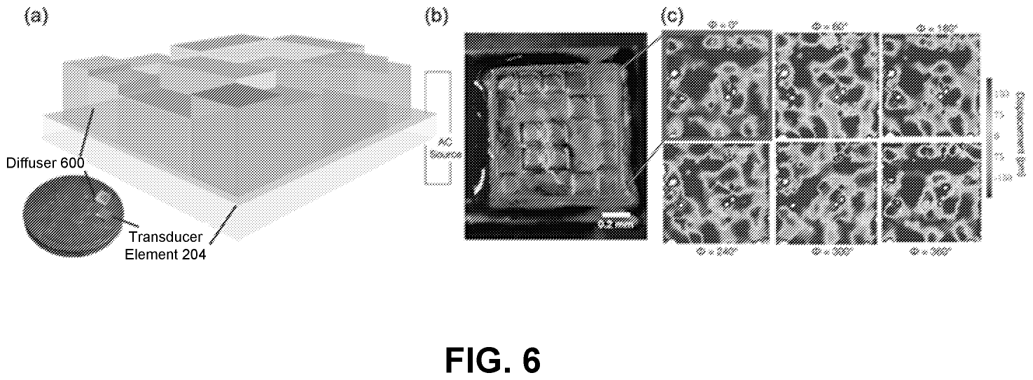

In some example embodiments, the stimulation apparatus 120 disclosed herein may include a diffuser, which may be disposed on a face of the transducer element 204, to produce spatiotemporally incoherent megahertz-order ultrasound. FIG. 6 depicts an example of the stimulation apparatus 120 in which a diffuser 600 is disposed on the face of the transducer element 204. Instead of being used to reduce coherent reflected sound (e.g., echoes), the diffuser 600 in this case is coupled with the sound generator itself, the transducer element 204. The diffuser 600 is ideally suited for sonogenetics as the diffuser 600 is nearly losslessly reduces the presence of regions of either high or low intensity within an enclosed cavity, in both in vitro assays and within the rodent skull for longer term applications.

Referring to FIG. 6(a), the diffuser 600 may be design based on Schröder's method of quadratic-residue sequences to determine well depth. For example, the diffuser 600 may include one or more wells machined in a substrate material, such as glass, using a KrF excimer laser system with a custom metal mask to restrict beam width. The machined depth of the pillars may be up to 309 μm. FIG. 6(b) shows an example of the resulting diffuser block, which may be bonded to the transducer element 204 operating in the thickness mode at 7 megahertz using an ultraviolet light-curable epoxy. A scanning laser Doppler vibrometer image of the diffuser face in the time domain is shown in FIG. 6(c) to exhibit phase differences corresponding to pillar heights (normalized autocorrelation >0.73).

The design of the Schröder diffuser is based on quadratic-residue sequences defined by sₙ=n², where n² where is the least non-negative remainder mod N, with N always an odd prime. One of the properties of this number sequence relevant to the design of an optimum diffuser is that both the Fourier transform of the exponential sequence rₙ=exp(i2πSₙ/N) and by extension the scattered wave produced by it have a constant magnitude expressed by Equation (1) below in which l=\sqrt{−1}.

$${\vert {R}_{m} \vert}^{2} = {\vert \frac{1}{N} {\sum }_{n = 1}^{N} {r}_{n} {e}^{- \frac{2 π mn}{N}} \vert}^{2} = \frac{1}{N} \qquad ( 1 )$$

This may then be used to define the wells' depths, d(xₙ>yₙ) corresponding to the number sequence. In one dimension, the depth of the nth well is given by Equation (2).

$${L}_{n} = \frac{π {cn}^{2} ( modN )}{N {ω}_{r}} \qquad ( 2 )$$

where ωᵣ is the design frequency, N is a prime number, and c is the speed of sound in the medium. Extending the concept of a diffuser defined per the above numerical sequence to two dimensions involves replacing n² in the above formula with n²+m², where m represents the number of wells in the second dimension. A representative image of a diffuser fabricated using a 2D sequence is shown as the diffuser 600 in FIG. 6.

While a 1D diffuser creates a uniform 2D pressure field, a 2D diffuser with varying well depths creates a uniform 3D pressure field. Ultrasound neuromodulation typically relies on frequencies in the 1-10 megahertz range and this requires submillimeter well depths as defined by Equation (2). Although structures based on the quadratic-residue sequence have been achieved at the macroscale in two dimensions and at the microscale in one dimension, it has not been achieved in 2D structures on the micron to submillimeter scale due to the lack of established fabrication techniques for these dimensions. Conventional photolithography is good for creating patterns that have the same depth or, at most, a few different depths. It becomes challenging when features of varying depths are desired because multiple photolithography and etching steps are required. Alternate approaches, including 3D or two-photon printing methods, are unable to produce acoustically low-loss structures with sufficient dimensional accuracy at these scales. In some example embodiments, these limitations are overcome by using an excimer laser to machine submillimeter pillars of varying heights in a substrate material (e.g., glass) in two dimensions. Significant phase correlation (normalized autocorrelation >0.73) with the machined geometry is apparent from a time-domain laser Doppler vibrometry scan shown in FIG. 6. The transducer element 204 in the example shown in FIG. 6 may be driven at its resonance frequency with a sinusoidal input power range of 0.5-2 watts and a peak pressure output of 0.6 MPa as measured with a fiber optic hydrophone.

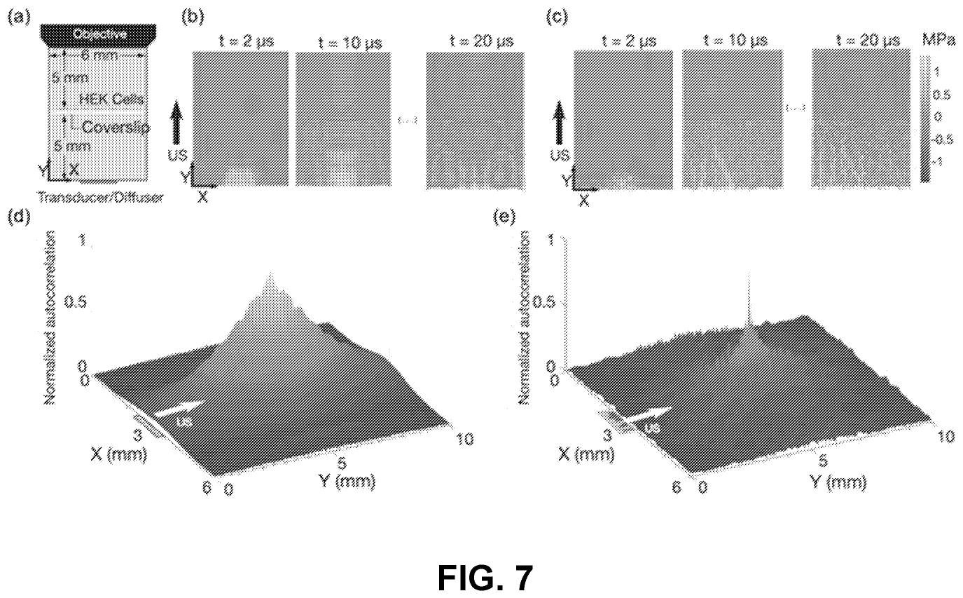

The benefit of using the diffuser 600 was considered using finite element analysis. The domain was chosen to mimic an experimental setup used for identifying ultrasound-sensitive ion channels in an in vitro setup. This includes an inverted fluorescence microscope with a custom perfusion chamber to house a coverslip and transducer. The simulation domain is illustrated in FIG. 7. Due to computational constraints, the simulation was modelled in two dimensions with 17 wells instead of the full 25-well system. The transducer element 204 and the diffuser 600 assembly were fixed at the bottom of the domain. A custom perfusion chamber that contains a slot for a coverslip was mounted over the ultrasound source. The transducer element 204 was coupled to the coverslip through water and there was a layer of media above the coverslip. The walls were defined to be rigid boundaries with an acoustic impedance Zᵢ=∞ such that the normal derivative of the total acoustic pressure

$$\frac{\partial {p}_{t}}{\partial n} = 0.$$

The diffuser 600 in the experimental setup includes of 17 elements, the heights of which were calculated from Equation (2). The coverslip serves as a solid boundary and allows the evaluation of the acoustic field in the closed domain below and the open domain above it, corresponding to the different boundary conditions assigned to the model.

The time variation of the pressure field with and without the diffuser 600 was evaluated. Several points in the fluid domain were chosen and the time evolution of the pressure field for the two cases was compared. A 2D autocorrelation was calculated in order to determine if there were any locations within the domain with coherence (echoes) or localized increases or decreases (constructive and destructive interference) in ultrasound intensity spatial and temporal patterns that form over the duration of the stimulus are represented by a 2D autocorrelation in FIG. 7. It is evident that there is both spatial and temporal periodicity with the transducer alone (see, e.g., FIG. 7(a)) that is greatly reduced when the diffuser is introduced (see, e.g., FIG. 7(b)). Videos of the sample autocorrelation in the domain over the stimulus duration show that there is greater autocorrelation over the duration of the stimulus without the diffuser 600. This indicates that the ultrasound field with the diffuser 600 is temporally aperiodic.

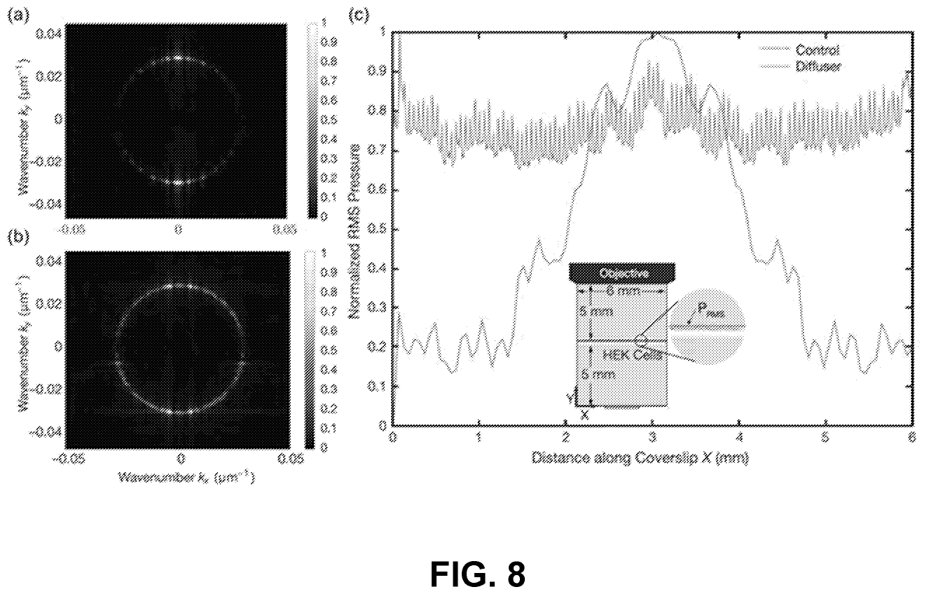

For the purpose of quantifying any changes to the diffraction at 7 megahertz through the inclusion of the diffuser, an isofrequency contour plot of the simulated data is provided in FIG. 8(a) without the diffuser 600 and in FIG. 8(b) with the diffuser 600. Without the diffuser 600, wave vectors are only present in the vicinity of kₓ=0, along the direction of propagation of the pressure wave in the medium: the Y axis. The angular spread is 20 on either side of the direction of propagation without the diffuser. Particularly, the majority of the wave can be seen to be propagating along the Y axis, with significant sidelobes immediately to the left and right and much smaller sidelobes slightly farther away. Including the diffuser 600 produces wave vectors beyond the main direction of propagation (see, e.g., FIG. 8(b)), with significant components oriented along directions from the Y axis (along kₓ) to the X axis (along k_{y}). The previously significant sidelobes remain significant, but are augmented by wave propagation beyond 45 in the XY plane. This indicates strong diffraction from the face of the transducer when including the diffuser. The root-mean-square (RMS) pressure was calculated to determine the temporal and spatial distribution of pressure 10 μm above the coverslip, as shown in FIG. 8(c). The inclusion of the diffuser 600 results in an even root-mean-square (RMS) pressure distribution along the coverslip, whereas the control case shows a fivefold variation of pressure across the coverslip face.

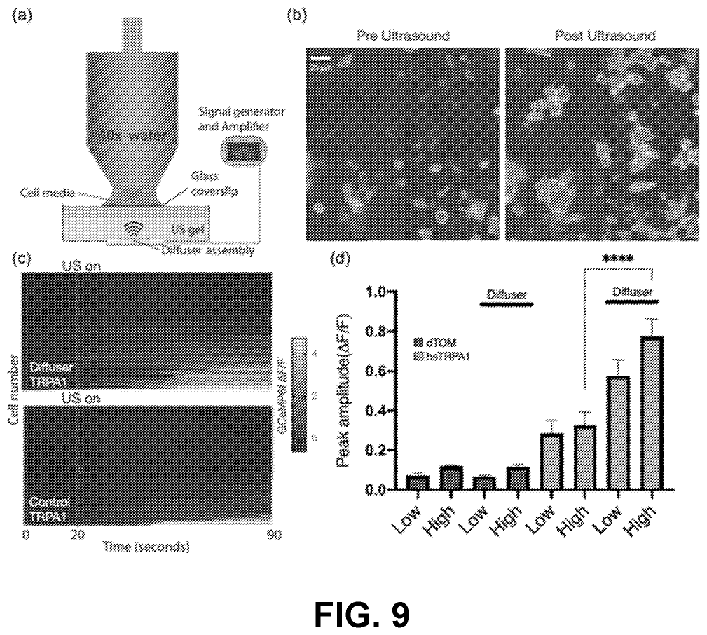

The testing setup to verify the effects of the diffuser 600 in vitro includes an upright optical imaging setup including an immersion objective, a custom perfusion chamber, and the diffuser assembly including the transducer element 204 coupled with the diffuser 600. The diffuser assembly and the testing setup are shown in FIG. 9(a). In this particular setup, the transducer element 204 may be formed from lithium niobate due to its relatively high coupling coefficient and zero hysteresis, which implies no heating from the piezoelectric material itself. Human embryonic kidney (HEK293) cells expressing GCaMP6f were transfected with hsTRPA1. Fluorescence changes

$$( \frac{Δ F}{F} )$$

were analyzed across four cases, with and without the channel, without the diffuser 600 (e.g., the transducer element 204 alone), and with the diffuser 600. Representative GCaMP6f images of HEK293 cells transfected with hsTRPA1 are shown in FIG. 9(b) and heat maps of fluorescence intensity with respect to time are presented in FIG. 9(c), with a clear increase in both the magnitude and number of cells being activated with the presence of the diffuser 600. Cells expressing hsTRPA1 and controls were tested at two different pressure amplitudes, 0.32 and 0.65 MPa, with the ambient pressure as the reference (zero) pressure. There was a consistent increase in fluorescence intensity with an increase in acoustic pressure for both the control and the hsTRPA1 condition, whether or not the diffuser 600 was present. As shown in FIG. 9(d), including the diffuser 600, increased the mean fluorescence amplitude by at least a factor of two for cells that had been infected with hsTRPA1 (p<0.0001). The application of ultrasound was also shown to have an effect on mouse primary cortical neurons. Neurons were infected with adeno-associated viral (AAV) vectors to express hsTRPA1 and a genetically encoded calcium indicator, GCaMP6f, or a control with only the calcium indicator. Ultrasound in this case triggered an increase in calcium uptake in both cases, with the hsTRPA1 neurons showing a greater number of activated cells in comparison to the control.

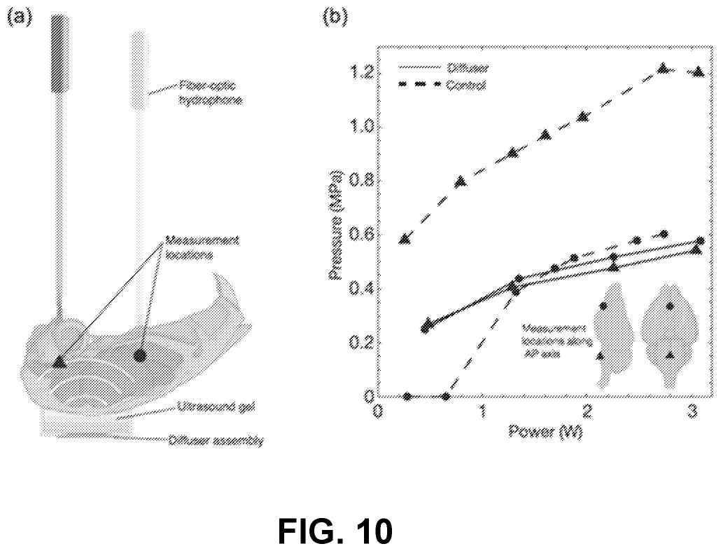

The uniform nature of the ultrasound field created by the diffuser 600 was also verified ex vivo in a mouse skull while keeping as much of the mouse skull intact during preparation as possible. Pressure measurements were taken at two different locations as indicated in FIG. 10 along the anterior-posterior axis, at the ventral surface of the pons and the ventral surface of the anterior olfactory bulb. With the diffuser 600, the pressure at both these locations was uniform, with minimal deviation between them and a uniform increase with input power to the transducer element 204. However, the transducer element 204 alone produced diverging values of pressure at these positions, so much so that the pressure at the pons (triangle) exceeded the pressure at the anterior olfactory bulb (circle) by a factor of 3 at an input power of 3 watts, yet fell below the hydrophone's minimum measurement value, 0.2 MPa, at the anterior olfactory bulb when using less than 1.25 watts of power. By contrast, when the transducer element 204 is coupled with the diffuser 600, minimal deviation in pressure values are observed at these locations, with pressure values ranging from 0.25 to 0.5 MPa at the ventral surface of both the pons and the anterior olfactory bulb. These brain regions were chosen not for their function, but because they were remote and would therefore be expected to exhibit standing-wave behavior with large variations in the acoustic pressure. Collectively, these results demonstrate that the diffuser 600 is capable of delivering uniform ultrasound fields in vivo in comparison to the transducer element 204 alone, thus enabling sonogenetic studies across large brain regions.

As noted, existing non- and minimally invasive techniques to stimulate brain regions, such as transcranial magnetic stimulation and transcranial direct current stimulation, offer poor spatial resolution. This is a problem for precisely targeting brain regions that have specific functions. Ultrasound-based stimulation enables targeting brain regions with submillimeter-scale accuracy. This precision can be achieved in different ways, either by using an array to focus ultrasound to a specific region or by using sonogenetics to engineer cells to locally be more sensitive to mechanical stimuli. The development of sonogenetics that started with the TRP4 channel has expanded to include a library of proteins that are sensitive to ultrasound stimuli at different ultrasound stimulation parameters. Examples include MSC, TREK, Piezo, and other TRP channels, all of which have been shown to be sensitive to ultrasound in vitro.

Nevertheless, as noted, a limitation with focused ultrasound is the alteration in the position and shape of the focal zone due to spatial variations in acoustic impedance. Sonogenetics is an attractive option because of the potential of having a toolkit of specific proteins that can be engineered to be sensitive to ultrasound stimuli at different frequencies or pressures. Current ultrasound transducers and how ultrasound interacts with the skull cavity are important limitations in translating sonogenetics into clinical practice. Standing waves in the skull cavity produce nodes and antinodes, each separated by one-half of the acoustic wavelength and responsible for pressure minima and maxima, respectively. This may lead to hemorrhage and heating in tissue as reported in past studies. One could attempt to overcome this issue by using broadband white noise to produce a spatiotemporally random acoustic field, but ultrasonic transducers are unable to provide such noise at pressures sufficient to elicit cellular responses. Considering the mechanical index for the ultrasound used with various examples of the stimulation apparatus 120 disclosed herein, which at below 0.15 is well below the U.S. Federal Drug Administration's clinical safety threshold index of 1.9 without microbubbles, cavitation and adverse heating effects are unlikely.

In some example embodiments, the transducer element 204 of the stimulation apparatus 120 may be coupled with the diffuser 600. In some cases, the diffuser 600 may be a microscale Schröder diffuser designed via computational analysis and fabricated with an excimer laser. The diffuser 600 may be configured to eliminate the spatiotemporally heterogeneous distribution of ultrasound by placing it upon the transducer element 204. The transducer element 204 alone was shown to produce standing waves in the absence of the diffuser 600. With the diffuser 600 in place, autocorrelation of the ultrasound field quantifies the elimination of the standing waves and consequent suppression of antinodes associated with potential tissue damage. The predictions of the simulation were verified in vitro using HEK293 cells and neurons that were transfected with a sonogenetic candidate, hsTRPA1.

Schröder's original diffuser design was to be used for diffusion of reflected sound in the far field of the source, not the near field. The essential distinction here is that near the transducer element 204 (e.g., in the near field), the acoustic field will exhibit a different distribution than acoustic fields in the far field away from the transducer element 204. In this case, the boundary between the near field and far field from the transducer element 204 may be defined as z=a²λ⁻¹, where a is the lateral size of the transducer element 204 and A is the wavelength. Accordingly, the far field of the transducer element 204 may begin 120 millimeters away from the transducer element 204. This is a far greater distance than the opposite side of the mouse skull, and so the entire system is in the near field of the transducer element 204. Existing efforts modify Schröder's diffuser design for optimal performance in the near field have been unsuccessful due to fabrication difficulties and modest improvements over the far field design. Contrastingly, the results associated with various examples of the stimulation apparatus 120 disclosed show that mounting the diffuser 600 with a Schröder based design on the transducer element 204 itself (e.g., as close to the source as is physically possible) is capable of yielding effective results.

It should be appreciated that development of sonogenetics in larger animal models—such as primates—will require ultrasound transducers that are capable of delivering an acoustic field that is spatially and temporally incoherent, a notable feature of various examples of the stimulation apparatus 120 disclosed herein. This ensures that the pressure in different regions of the target organ (e.g., brain, pancreas, and/or the like) is uniform over the stimulus duration, thus eliminating the aberrations in the acoustic field due to the skull cavity. Functionalization of specific brain regions using ultrasound-sensitive proteins can offer submillimeter spatial precision. Localization of sonogenetic proteins in combination with an acoustic field provided by a diffuser assembly will also ensure that the observed neuromodulatory effects are solely due to ultrasound activation of targeted regions of tissue and not due to the confounding effects of reflection or interference from the geometry of the skull.

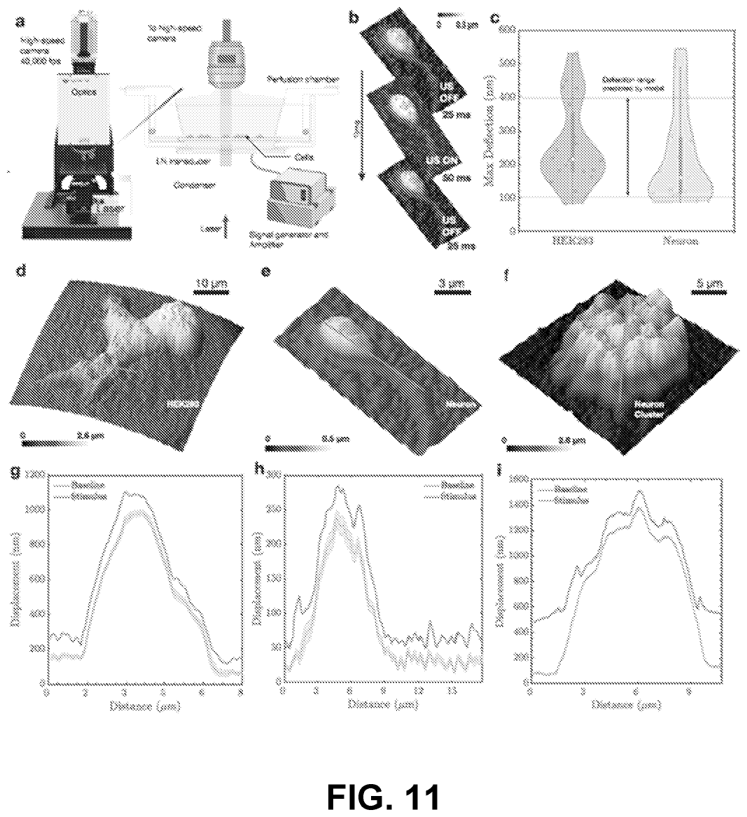

As noted, the actual sonogenetic and ultrasonic-to-chemical action mechanisms associated with ultrasonic cellular stimulation have eluded in vitro and in vivo analysis, yet such an understanding may be fundamental to a precise prescription, application, and control of ultrasonic cellular stimulation in a clinical setting. In some example embodiments, transmission high-speed digital holographic microscopy (DHM), which measures transparent media based on quantifying phase disparities induced by the measured sample, may be used to analyze the ultrasound induced cell membrane dynamics. For example, transmission high-speed digital holographic microscopy (DHM) may operate by comparing phase differences induced in the coherent light transmitted through the sample with reference light traversing an unobstructed path. Digital holographic microscopy has several advantages in comparison to conventional microscopic techniques. Numerical processing of the wavefront transmitted through the sample permits simultaneous computation of intensity and phase distribution. The holographic measurements also make it possible to focus on different object planes without relative movement between the stage and the lens and enables numerical lens aberration correction. The unique digital holographic microscopy system disclosed herein operates at high frame rates (40,000 frames per second) and includes the custom-built perfusion chamber with a built-in ultrasound transducer shown in FIG. 11(a). A heated stage keeps the media at a constant temperature over the duration of the recording. The system reconstructs phase images of cells that are then analyzed to determine the baseline profile (prior to ultrasound), during exposure to ultrasound, and afterward. This enables an accurate visualization of the maximum displacement of the membrane from the mean position under the influence of ultrasound.

The measurements of apical cellular membrane deflection due to ultrasound includes a 25-millisecond baseline recording, followed by a 50-millisecond ultrasound stimulus, and a 25-millisecond post-stimulus dwell (see, e.g., FIG. 11(b)), leading to a median deflection of 214 nanometers for human embryonic kidney (HEK293) cells and 159 nanometers for neurons, with a range of 100 to 550 nanometers m across the two tested cell types (see, e.g., FIG. 11(c)). Sample reconstructed phase images of HEK293 cells, neurons, and neuronal clusters are shown in FIGS. 1(d) through (f). The baseline deflection for these samples, including a 95% confidence interval, had a range of ±20 nanometers, inclusive of both random thermal fluctuations across the cell membrane and potential noise introduced to the system due to the imaging arrangement (see, e.g., FIGS. 11(g) through (i)). Sample displacement baseline membrane profiles are illustrated in FIGS. 11(g) through (h) for HEK293 cells and neurons, and FIG. 11(i) represents the deflection profile for a cluster of neurons. The cluster was imaged to confirm deflection in a group of neurons and help provide insight into the in vivo mechanisms of activation. Results from the neuronal cluster show that the magnitude of deflection remains roughly the same for a group of cells as for a single neuron. The larger deflection at the edges of the cluster is due to the neurons at the edges being less constrained in comparison to the ones in the center.

In addition to membrane deflection during the generation of action potentials, the converse phenomenon of membrane deflection leading to the generation of action potentials may be explored using various examples of the digital holographic microscopy (DHM) system described herein. In particular, compared to other imaging techniques, examples of the digital holographic microscopy (DHM) system disclosed herein provides unparalleled spatiotemporal capabilities. Overall, the experimental setup confirmed that ultrasound stimulation induces cell membrane deflection for cells adherent to a coverslip. These results are further applied to generate a deflection model 115 which, as shown in FIG. 1, may be deployed as a part of the stimulation controller 110 to control the operations of the stimulation apparatus 120.

Based upon the results from the experiments, with cells cultured on a surface and surrounded by media, the membrane is assumed to be fixed at the periphery. A similar case occurs in vivo, where the extracellular matrix holds individual cells in place and provides anchoring locations for sections of the membrane. Cellular anchoring is important because it imposes a characteristic distance over which the range of permissible deflection wavemodes may occur. Its deflection is restricted in the analysis to a single direction, perpendicular to the plane of the membrane and parallel to the direction of propagation of sound. In some cases, the deflection model 115 may ignore the restoring effects of the actin cytoskeleton, which is difficult to estimate and likely plays an important role in restoring the membrane to its original equilibrium position.

The stimulus provided to the cells is in the form of a sinusoidal burst, which is a short-term continuously oscillating ultrasound signal of constant amplitude and frequency. In a burst, a sinusoidal electrical signal is typically applied across the piezoelectric material used in a transducer (e.g., the transducer element 204 of the stimulation apparatus 120), which transforms this signal into a sinusoidally varying pressure field in the fluid medium at the frequency of excitation. Instead of modeling the ultrasound as a step increase in hydrostatic pressure from zero to a fixed positive value at t=0, the deflection model 115 may model ultrasound as a burst signal oscillating at the ultrasound frequency. An analytical solution for the slower time scale of the membrane mechanics may then be found in response to this harmonic ultrasound excitation. This solution is then used in the deflection model 115 to produce the solution for the deflection of the fixed membrane, resolving the discrepancy between the timescales of ultrasonic stimulation (≈0.1 μs) and the experimentally verified membrane deflection occurring on the order of milliseconds. This hybrid approach was chosen because a numerical simulation of the entire phenomena from ultrasound to membrane deflection would be extremely difficult due to the vastly different spatiotemporal scales, even with state-of-the-art computational resources. Finally, the corresponding hydrostatic pressure is discarded here, because it is orders of magnitude lower than the ultrasonic radiation pressure.

The damped wave equation describing the deflection, u, of the membrane in response to ultrasonic pressure, P_{US}, is written as

$$ρ {\partial }_{t}^{2} = 2 η \frac{{\partial }^{3} u}{\partial {x}^{2} \partial t} + ( 2 γ {\partial }_{x}^{2} u + {P}_{US} ) ( \frac{π}{d} ) \qquad ( 3 )$$

wherein ρ and η are the dynamic viscosity and density of the surrounding fluid, both assumed to be the same as water as used in prior studies, γ is the surface tension between the membrane and media; and d is the characteristic length of the membrane between anchor points. Equation (3) was solved by the method of eigenfunction expansion. FIG. 11 provides results representative of the analysis, with a 1 MPa pressure supplied to the membrane using a 7 megahertz transducer in the form of a sine wave over a period of 5 milliseconds. The mechanical index for the parameters listed in this experimental setup is 0.37, well below the oft-cited mechanical index threshold for cavitation onset of 0.7 in bubble-perfused tissue. Since no bubbles were used in this setup, the U.S. Federal Drug Administration's mandated clinical safety threshold index of 1.9 without introduced microbubbles is more appropriate. These data suggest that we are unlikely to cause cavitation and cell viability remains unaffected.

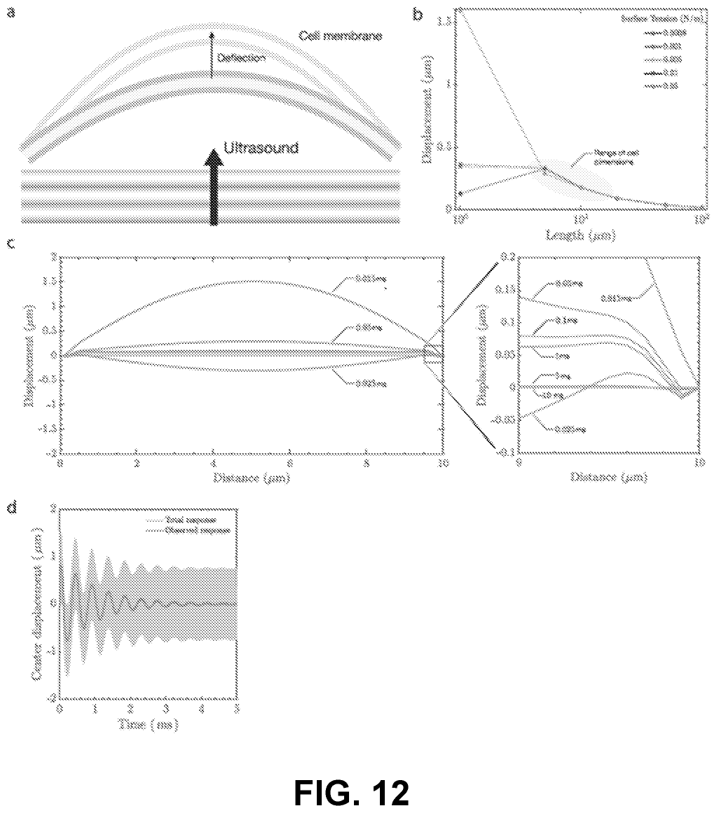

As shown in FIG. 12(a), maximum membrane deflection occurs when the ultrasound stimulus is applied, followed by decay due to viscous losses to the host medium. The magnitude of deflection depends on the stimulation frequency and peak pressure, with lower frequencies and higher pressures producing greater membrane deflection. The critical parameters that influence the deflection magnitude are the characteristic membrane anchor length and surface tension, as shown in FIG. 12(b). The deflection predicted by the deflection model 115 for dimensions relevant to the size of a cell are between 100 and 400 nanometers, irrespective of the value of surface tension for an anchor length ranging from 5-20 μm based on the average size of the soma and average diameter of HEK293 cells. When membrane deflection due to a range of surface tension values reported in the literature was modeled, maximum membrane deflection was predicted to occur at the midpoint of the axisymmetric membrane model. This is portrayed in FIG. 12(c), which provides graphical “snapshots” of the ultrasonically-forced membrane over time. The closed-form displacement solution to Equation (3) provides a link to the fast ultrasonic timescales (on us order, or, total response) to phenomena occurring at observable timescales (on ms order, or, observed response), as shown in FIG. 12(d). The character of the membrane “slow time” response—that is, its ability (or lack thereof) to sustain oscillations—is governed by the value of the Ohnesorge number, Oh. The term is defined in this way because the membrane oscillations typically occur slowly (e.g., at a frequency far less than the incident ultrasound).

The nondimensional parameter Oh characterizes the importance of dissipative viscous forces relative to the combined interaction of conservative inertial and surface tension forces. In other words, Oh characterizes, on average, the extent to which the membrane dissipates or conserves mechanical energy. Typical Oh values for neurons range from ≈0.06 to ≈0.45 based on values of surface tension, viscosity, and membrane length considered in this work. This implies that inertial and surface tension forces dominate over viscous forces: the slow time membrane response is characteristically oscillatory. This behavior results from the membrane's tendency toward retaining mechanical energy in the form of sustained oscillations when Oh √\sqrt{2/π}≈0.8. This is explicitly derived in the detailed analysis and suggests that the slow time oscillations of the ultrasonically actuated membrane is implicated in the changes in the membrane capacitance as detailed in the following sections.

To model the electrical output of a neuron under the influence of ultrasound, a modified version of the original Hodgkin-Huxley equations is first used.

$$\frac{{dv}_{m}}{dt} = - \frac{1}{{c}_{m}} [ {I}_{app} + {I}_{Na} + {I}_{Kd} + {I}_{M} + {I}_{leak} ] \qquad ( 4 )$$

In this equation, the membrane potential of the neuron, Vm, changes over time with respect to the membrane capacitance, Cm, and the underlying currents, Iₐₚₚ, I_{Na}, I_{Kd}, I_{M}, and I_{leak. At rest v}_{ₘ}_{=-71.9 mV}, is the well-known membrane potential of the cell and, notably, the action potential generation is controlled by the presence of an applied current, Iₐₚₚ, while the other currents are based on the membrane morphology and chemistry. The increase of Iₐₚₚ beyond a certain threshold produces spiking behaviour typical of neurons. The capacitance, (′m, may also fluctuate due to a morphological change in the membrane. Such a modification is not modeled in the original representation of this equation, but it may be included. The voltage change as described in Equation (4) includes a time-dependent capacitive current

$${I}_{app} ≡ {V}_{m} \frac{{dC}_{m}}{dt} ,$$

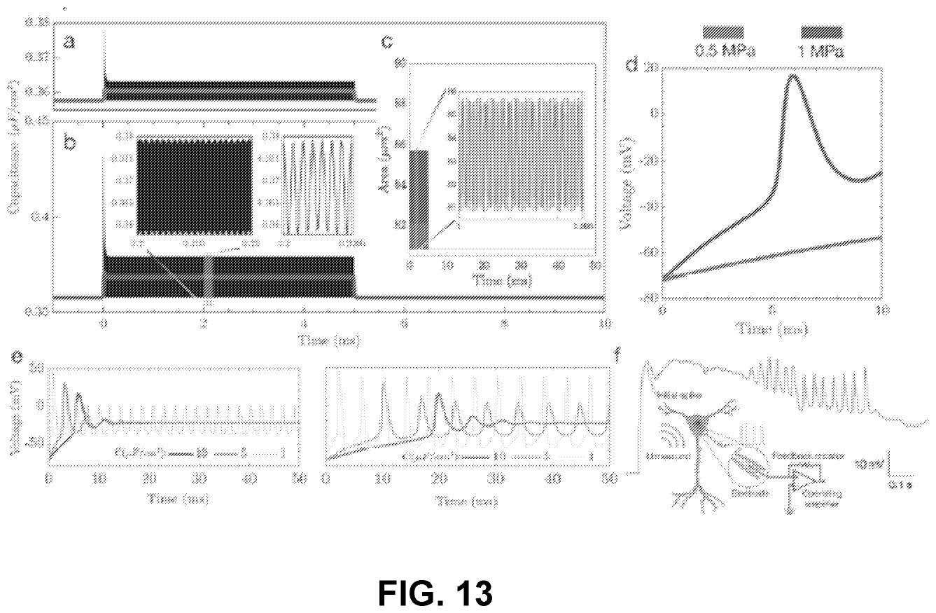

With this included in Equation (4), it is possible to solve the differential equation for the voltage and gating variables while incorporating the capacitance change due to membrane deflection. Membrane deflection is constrained to a certain extent due to parts of the cell that are adherent to the substrate or the extracellular matrix. This causes an increase in area between the adherent locations and with sufficient deflection, this produces a depolarization across the membrane. The value of the transmembrane voltage is dependent on the magnitude and duration of the applied stimulus. FIG. 13 indicates the change in capacitance due to 6.72 megahertz ultrasound at 0.5 (FIGS. 13(a)) and 1 MPa (FIG. 13(b)) with the corresponding area fluctuations that bring about the change in capacitance represented in FIG. 13(c). In order to compute the time dependent area variation, the slow time output of Equation (3) is extracted for use with the axisymmetric area integral. The capacitance of the membrane is then determined by treating it as a dielectric between charged surfaces. This produces a slow time capacitive response, bearing an order of magnitude equivalence to the ion channel relaxation times in the modified Hodgkin-Huxley model.

The stimulus of 1 MPa results in depolarization as indicated in FIG. 13(d), while the lower pressure does not result in the generation of an action potential over the stimulus duration. Reported values of baseline membrane capacitance have been shown to vary, and longer stimuli will result in the generation of action potentials as a cumulative effect of capacitance change over the duration of the stimulus. FIG. 13(e) represents transmembrane voltage changes for a stimulus of 50 milliseconds. Depolarization takes place in both cases. However, initial spikes are delayed by up to 20 milliseconds in the lower pressure case, indicating the need for increased stimulus durations for lower pressures. The deflection model 115 also shows a lower spike frequency for the 0.5 MPa case in comparison to 1 MPa. The simulation output of the deflection model 115 for the lower pressure and longer stimulus duration case were verified experimentally using voltage clamp electrophysiology (see, e.g., FIG. 13(f)) and shows an initial spike corresponding to the delivery of the ultrasound stimulus, followed by oscillations.

The deflection model 115 may represent how ultrasound results in membrane deflection and eventually leads to transmembrane voltage changes. At the outset, real-time membrane deflection due to ultrasound may be demonstrated using high-speed digital holographic microscopy (DHM) imaging. The Hodgkin-Huxley equations, which are a set of phenomenological equations describing action potential generation in a squid axon and are one of the most important neuronal models, are leveraged. However, observations of mechanical deflection accompanying action potentials show that the underlying assumptions of the Hodgkin-Huxley model may need to be revisited, as there are mechanical phenomena involved. In the context of ultrasound neuromodulation, the deflection model 115 disclosed herein presents insights into the generation of action potentials due to mechanical deflections and is theoretically supported by other models. The deflection due to the applied ultrasound stimulus results in a net area change of the membrane between the two pin locations that represent an adherent cell. The area changes take place elastically while maintaining constant volume. This results in a change in capacitance that, when incorporated in the Hodgkin-Huxley model, results in transmembrane voltage changes. Capacitance of the membrane can be modeled using an expression for a parallel plate capacitor, and an increase in area results in a proportional increase in capacitance.

The deflection model 115 does not take into account restoring effects of the actin cytoskeleton, whose influence will lower the membrane de-flection and cause the inner leaflet to deflect less than the outer leaflet. However, this cannot account for the ≈100 nm deflection observed in experiments, and only plays a minor role in bringing about capacitance changes according to previous studies. The deflection model 115 and the use of high-speed digital holographic microscopy (DHM) imaging present opportunities for exploring the influence of ultrasound on native neurons and HEK293 cells. A combination of fluorescence imaging with digital holographic microscopy can be used to image focal adhesions and cells that have been engineered to express membrane proteins that are sensitive to ultrasound stimuli, in other words using sonogenetics. At a cellular level, there are two proposed models for the activation of a mechanically-gated ion channel: the force from lipid model and the force from filament model. The force from lipid model proposes that changes in membrane tension or local membrane curvature result in opening or closing of channels. In the force from filament model, the stimulus is transferred to tethers that connect the membrane to the cytoskeleton. Conformational changes in the tethers result in opening or closing of the channel. In reality, both models play a part in opening and closing a given channel.

Although it is difficult to estimate the relative contribution of these mechanisms, it is possible to estimate the deflection of the cell membrane as highlighted in the preceding sections. This is of particular significance given the membrane bound proteins such as TRPAI, MsCL, Piezo, and their interaction with the action network. Disruption of the actin cytoskeleton has been shown to reduce mechanosensitive activity of such ion channels and it is possibly due to decreased separation between the leaflets of the bilayer when the action network is disrupted. In addition to quantifying the deflection due to mechanosensitive proteins, there is potential to quantify the forces on the cell due to ultrasound using Förster resonance energy transfer force sensors.

In some example embodiments, the deflection model 115 also predicts the generation of action potentials from capacitive changes that occur when the adherent cell is exposed to ultrasound. Charge across the membrane is maintained by a gradient in ion concentration across the cell membrane, with Na+ ions on the outside and Cl— ions on the inside, resulting in a net negative resting potential. As the membrane deflects, it is partially constrained by the adherent regions, resulting in an increase in area of the membrane between the adherent locations. An increase in the area of the membrane directly increases its capacitance.

Transmembrane voltage changes are demonstrated for a pressure of 0.5 MPa and a pressure of 1 MPa. The observation is that voltage changes only take place for the higher pressure case for lower stimulus durations, thus defining a pressure threshold dependent upon the duration of stimulus. The influence of longer stimulus durations on the generation of action potentials is also investigated for different values of baseline capacitance. As verified by a current clamp electrophysiology study in the whole cell configuration, increased stimulus durations even at lower pressures result in action potential generation, though with lower spike rates.

One of the limitations with performing single cell current clamp electrophysiology while using ultrasound at amplitudes sufficient to drive a physiological response is the loss of a seal between the membrane and the patch pipette due to the membrane's deflection. There are, however, reports of current clamp electrophysiology results with ultrasound using microbubbles and at much higher frequencies or with devices. In each of these three cases, there is reason to believe that while the stimulation techniques or device may work for in vitro work, they will not be suitable for in vivo work. One potential way to overcome this issue would be to perform electrophysiological recordings for cells encased in matrigel that would limit the movement of the recording pipette with respect to the membrane.

In some example embodiments, identifying the mechanisms underlying ultrasound neuromodulation offers valuable insight into the underlying effects of ultrasound on cell membranes, as well as insight into how these effects translate to transmembrane voltage changes. The predictions of the deflection model 115 were confirmed using a novel, high-speed imaging technique. Leveraging this real time visualization and quantification of membrane deflection, the deflection model 115 may enable a prediction of the depolarization due to the imposed ultrasound stimulus.

In some example embodiments, the deflection model 115 may be configured to the model membrane deflection and transmembrane voltage changes induced by ultrasound stimulus applied, for example, by the stimulation apparatus 120. As the pressure wave propagated through the fluid and contacted the adherent cell, the region of the cell membrane between adhesion zones deflected. This deflection led to a change in area of the membrane and causes a capacitance change. The two-dimensional model assumed that the membrane had a known value of surface tension. The membrane was surrounded by a fluid, assumed to have the properties of water in this case. The vertical displacement of the membrane was approximated to be equal to the displacement of the fluid just above the membrane. The start was with a simplified version of the Navier-Stokes equation

$$ρ ( {\partial }_{t} v + v \cdot ∇ v ) = η {∇}^{2} v - ∇ P \qquad ( 5 )$$

where ρ and η are the density and viscosity of water, respectively. The expression ∇P is the pressure gradient and v is the velocity. In Equation (5), the convective acceleration is v·∇v=0 as the flow is unidirectional in z and the fluid is assumed to be incompressible. The membrane was symmetric in x and y, allowing the viscous term to be simplified as ∂ₓv_{z}=∂_{y}v_{z}. What was left were

$$ρ {\partial }_{t} {v}_{z} = 2 η {\partial }_{x}^{2} {v}_{z} - ∇ P \qquad ( 6 )$$

The net pressure gradient in this case is a function of the time dependent pressure in the fluid due to ultrasound and the surface tension of the membrane, which resists deformation

$$∇ P = - ( 2 γ {\partial }_{x}^{2} u + {P}_{US} ) π d \qquad ( 7 )$$

where u is the displacement in z and P_{US} is the pressure due to an ultrasound source, typically acting in the form of a sinusoidal pulse, P_{US}=P₀ sin(ωt), where ω=2πf. By contrast, other models at this point chose to represent the ultrasound as a step change in the pressure, from a static, zero relative pressure to a static positive value at time t=0 well below the pressure amplitudes used in experimental studies, typically 1 kPa to 1 MPa. Such representations may be numerically attractive but difficult to reconcile with the harmonic oscillatory pressure delivered by the transducer. In the absence of an analytical solution for the ultrasound propagating through the medium and membrane, one would be forced to numerically represent the megahertz-order sinusoidal signal with sufficiently small spatiotemporal step sizes to satisfy the Nyquist criterion, and do so for at least several hundred milliseconds to determine the response of the cell membrane to the ultrasound pressure oscillation, producing very large models with many millions to billions of temporal steps for a single solution. Consequently, past studies were understandably forced to make spurious approximations to avoid impossibly prohibitive computation times.

Substituting this into Equation (6) produced a partial differential equation for the displacement of the membrane driven by ultrasound

$$ρ {\partial }_{t}^{2} u = 2 η \frac{{\partial }^{3} u}{\partial {x}^{2} \partial t} + ( 2 γ {\partial }_{x}^{2} u + {P}_{US} ) ( \frac{π}{d} ) \qquad ( 8 )$$

The boundary conditions are the clamped conditions at the ends of the membrane and the initial displacement condition

$$\begin{matrix}u ( 0 , t ) = 0 & ( 9 ) \\ u ( d , t ) = 0 & ( 9 a ) \\ u ( x , 0 ) = \frac{{P}_{0} x ( d - x )}{4 γ} ≡ {u}_{0} ( x ) & ( 9 b ) \\ \partial {t}_{u ( x , 0 ) = 0} & ( 9 c )\end{matrix}$$

If hydrostatic pressure is included, the initial condition for membrane displacement may be found by solving P₀+2γ ∂ₓ²u=0. The general solution to partial differential Equation (8) was obtained with the method of eigenfunction expansion, as outlined further on. This was achieved using an orthogonal eigenbasis

$${ϕ}_{n} ( x ) = sin ( \sqrt{{χ}_{n}} x ) \qquad ( 10 )$$

where χₙ=(nπ/d)² corresponds to the nth wavemode for a membrane with diameter d. Expanding u gives

$$u ( x , t ) = {Σ}_{n} {u}_{n} ( x , t ) = {Σ}_{n} {h}_{n} ( t ) {ϕ}_{n} ( x ) \qquad ( 11 )$$

Accordingly, the even modes vanish and n=2 k+1 may be written, and k∈Z≥0 where Z is an integer set. Substituting this expression into Equation (6), one has

$${Σ}_{n} ( {\ddot{h}}_{n} + {c}_{1} {χ}_{n} {\dot{h}}_{n} + {c}_{0} {χ}_{n} {h}_{n} ) {ϕ}_{n} ( x ) = f ( t ) \qquad ( 12 )$$

where c₁=2n/ρ and c₀=2πγ/ρd, are written in terms of the density of the surrounding fluid, ρ; the viscosity of the surrounding fluid, η; the surface tension along the fluid-membrane interface, γ; and the membrane diameter, d. By multiplying both sides by ϕₘ(x) (with m∈Z⁺), integrating over x from 0 to d, and then leveraging the orthogonality of sines, it was found that the time-dependent component for the nth eigenmode satisfied the second-order ordinary differential equation

$${\ddot{h}}_{n} + {b}_{1 , n} {\dot{h}}_{n} + {b}_{0 , n} {h}_{n} = {\hat{f}}_{n} ( t ) \qquad ( 13 )$$

where b_{1,n}=c₁χₙ, b_{0,n}=c₀χₙ and

$${\hat{f}}_{n} ( t ) = \frac{2}{d} {\int }_{0}^{d} {ϕ}_{n} ( x ) f ( t ) dx = \frac{2 ( 1 - {( - 1 )}^{n} )}{n π} f ( t ) \qquad ( 14 )$$

The means for obtaining a solution to equations of the form Equation (13) is known. The homogeneous solution and its coefficients are given by

$${h}_{n}^{( h )} ( t ) = {a}_{+ , n}^{( h )} {e}^{{r}_{+ , n} t} + {a}_{- , n}^{( h )} {e}^{r - , nt} \qquad ( 15 )$$

where the coefficients a_{+,n}^{(h)} and a_{-,n}^{(h)} are −, n are

$$\begin{matrix}{a}_{+ , n}^{( h )} = \frac{{r}_{- , n}}{{r}_{- , n} - {r}_{+ , n}} {h}_{n} ( 0 ) & ( 16 a ) \\ {a}_{- , n}^{( h )} = \frac{{r}_{+ , n}}{{r}_{+ , n} - {r}_{- , n}} {h}_{n} ( 0 ) & ( 16 b )\end{matrix}$$

The inhomogeneous solution is

$${h}_{n}^{( i )} ( t ) = \frac{1}{{r}_{+ , n} - {r}_{- , n}} ( {e}^{{r}_{- , n} t} {𝒥}_{- , n} ( t ) - {e}^{{r}_{+ , n} t} {I}_{+ , n} ( t ) ) \qquad ( 17 )$$

$$where$$

$${I}_{\pm , n} ( t ) = {\int }_{0}^{t} {e}^{- {r}_{\pm , n} τ} \hat{f} ( τ ) d τ \qquad ( 18 )$$

The total waveform solution was then numerically implemented by taking a finite-term approximation of Equation (11).

The change in area, A, of the membrane then be calculated once the time-dependent membrane deflection is obtained

$$A = {\int }_{0}^{d} 2 π \sqrt{( 1 + {( {\partial }_{x} u )}^{2} )} dx \qquad ( 19 )$$

By extension, this allowed to determine the change in membrane ca-pacitance, C, due to the area change

$$C = \frac{{ϵ}_{0} ∈ A}{L} \qquad ( 20 )$$

where it was regarded that the membrane was a dielectric between two charged surfaces. In this case, L is the thickness of the bilayer and has values between 4 and 9 nanometers, and the relative permittivity, e, has a value of 2.

The above value of capacitance change was coupled with the modified Hodgkin-Huxley neuronal model, where the capacitive current is defined as

$${I}_{app} ≡ {V}_{m} \frac{{dC}_{m}}{dt} .$$

This model contained a voltage-gated sodium current and delayed-rectifier potassium current to generate actions, a slow non-inactivating potassium current to recapitulate the spike-frequency adaptation behaviour seen in thalamocortical cells, and a leakage current.

Equation (21) defines the voltage-gated Na⁺ current where g̅_{Na}=56 mScm⁻² is the maximal conductance and E_{Na}=50 mV is the Nernst potential of the Na⁺ channels. The parameter Vₜₕ=−56.2 mV sets the spike threshold

$${I}_{Na} = {\overline{g}}_{Na} \cdot {m}^{3} \cdot h \cdot ( {V}_{m} - {E}_{Na} ) \qquad ( 21 )$$

where the gating variables m and h vary with time according to

$$\begin{matrix}\frac{dm}{dt} = {α}_{m} \cdot ( 1 - m ) - {β}_{m} \cdot m & ( 22 a ) \\ \frac{dh}{dt} = {α}_{h} \cdot ( 1 - h ) - {β}_{h} \cdot h & ( 22 b ) \\ {α}_{m} = \frac{- 0.32 \cdot ( {V}_{m} - {V}_{th} - 13 )}{exp [ - ( {V}_{m} - {V}_{th} - 13 ) / 4 ] - 1} & ( 22 c ) \\ {β}_{m} = \frac{0.28 \cdot ( {V}_{m} - {V}_{th} - 40 )}{exp [ ( {V}_{m} - {V}_{th} - 40 ) / 5 ] - 1} & ( 22 d ) \\ {α}_{h} = 0.128 \cdot exp [ - ( {V}_{m} - {V}_{th} - 17 ) / 18 ] & ( 22 e ) \\ {β}_{h} = \frac{4}{1 + exp [ - ( {V}_{m} - {V}_{th} - 40 ) / 5 ]} & ( 22 f ) \\ {I}_{Kd} = {\overline{g}}_{Kd} \cdot {n}^{4} \cdot ( {V}_{m} - {E}_{K} ) & ( 23 )\end{matrix}$$

where g̅_{Kd}=6 mScm⁻² is the maximal conductance of the delayed-rectifier K⁺ channels and EK=−90 mV is the Nernst potential of the K⁺ channels, and with n evolving over time as

$$\begin{matrix}\frac{dn}{dt} = {α}_{n} \cdot ( 1 - n ) - {β}_{n} \cdot n & ( 24 a ) \\ {α}_{n} = \frac{- 0.032 \cdot ( {V}_{m} - {V}_{th} - 15 )}{exp [ - ( {V}_{m} - {V}_{th} - 15 ) / 5 ] - 1} & ( 24 b ) \\ {β}_{n} = 0.5 \cdot exp [ - ( {V}_{m} - {V}_{th} - 10 ) / 40 ] & ( 24 c )\end{matrix}$$

A slow non-inactivating K⁺ current may be defined as

$${I}_{M} = {\overline{g}}_{M} \cdot p \cdot ( {V}_{m} - {E}_{K} ) \qquad ( 25 )$$

where g̅_{M}=0.075 mScm⁻² is the maximal conductance and τₘₐₓ=608 ms is the decay time constant for adaptation of the slow non inactivation K+channels. The parameter p is such that

$$\begin{matrix}\frac{dp}{dt} = \frac{{p}_{∞} - p}{{τ}_{p}} & ( 26 a ) \\ {p}_{∞} = \frac{1}{1 + exp [ - ( {V}_{m} + 35 ) / 10 ]} & ( 26 b ) \\ {τ}_{p} = \frac{{τ}_{max}}{3.3 \cdot exp [ ( {V}_{m} + 35 ) / 20 ] + exp [ - ( {V}_{m} + 35 ) / 20 ]} & ( 26 c )\end{matrix}$$

The leakage current is

$${I}_{Leak} = {\overline{g}}_{Leak} \cdot ( {V}_{m} - {E}_{Leak} ) \qquad ( 27 )$$

where g_{Leak}=0.0205 mScm⁻² is the maximal conductance and E_{Leak}=−70.3 mV is the Nernst potential of the non-voltage-dependent, nonspecific ion channels. The following initial conditions were set for the gating terms

$$\begin{matrix}{m}_{0} = \frac{{α}_{m}}{{α}_{m} + {β}_{m}} & ( 28 a ) \\ {h}_{0} = \frac{{α}_{h}}{{α}_{h} + {β}_{h}} & ( 28 b ) \\ {n}_{0} = \frac{{α}_{n}}{{α}_{n} + {β}_{n}} & ( 28 c ) \\ {p}_{0} = {p}_{∞} & ( 28 d )\end{matrix}$$

Equations (21)-(26) were solved with initial conditions (28) to obtain the transmembrane voltage change of a neuron when subjected to ultrasound stimuli. A better understanding of the membrane wave propagation can be obtained by considering the decay transience of the constituent wavemodes within the context of the solution to Equation (14). Each wavemode will have a solution of the form

$${h}_{n} ( t ) = {h}_{n}^{( k )} ( t ) + {h}_{n}^{( i )} ( t ) \qquad ( 29 )$$

where hₙ^{(h)} is the homogeneous solution and hₙ⁽ⁱ⁾ is the inhomogeneous solution for the forced wavemode propagation initialized from zero initial conditions? The general form of the former can be used to characterize the decay transience

$${h}_{n}^{( h )} ( t ) = {a}_{+ , n}^{( h )} {e}^{{r}_{+ , n} t} + {a}_{- , n}^{( h )} {e}^{{r}_{- , n} t} \qquad ( 30 )$$

where the coefficients a_{±,n}^{(h)} are determined by the initial conditions and r±n are the eigenvalues of the left side of Equation (11) (the roots of the characteristic equation) as

$${r}_{\pm , n} = - \frac{1}{2} ( {b}_{1 , n} \pm \sqrt{{b}_{1 , n}^{2} - 4 {b}_{0 , n}} ) \qquad ( 31 )$$

Then the discriminant determines the character of the wavemode

$${b}_{1 , n}^{2} - 4 {b}_{0 , n} \{ \begin{matrix}> 0 , & {r}_{\pm , n} ∈ , & two distinct roots \\ = 0 , & {r}_{\pm , n} ∈ , & two degenerate roots \\ < 0 , & {r}_{\pm , n} ∈ ℂ , & two conjugate roots\end{matrix} \qquad ( 32 )$$

The physical conditions for degeneracy required an exacting degree of marginality rarely (if ever) encountered in real systems, so that this solution type may be safely ignored (degeneracy corresponds to algebraic growth at small times that was mediated by exponential decay at longer times).

Rewriting the conditions (32) in terms of physical parameters, it was found that

$$n \{ \begin{matrix}> \sqrt{\frac{2}{π}} {Oh}^{- 1} , & {r}_{\pm , n} ∈ , & strictly decaying wavemode \\ < \sqrt{\frac{2}{π}} {Oh}^{- 1} , & {r}_{\pm , n} ∈ ℂ , & oscillatory decaying wavemode\end{matrix} \qquad ( 33 )$$

$$where$$

$$Oh = \frac{η}{\sqrt{ργ d}} \qquad ( 34 )$$

is the Ohnesorge number characterizing the balance between the dissipative viscous effects and the conservative effects resulting from interaction between inertia and surface tension. There exists a condition for oscillation of the unforced membrane and this condition is Oh<\sqrt{2/π} When Oh<\sqrt{2/π}, no oscillatory unforced wavemodes are permitted and the unforced membrane will not oscillate. When the condition is satisfied, it was observed that oscillation can be attributed exclusively to wavemodes with the “smallest” mode numbers, and that these will always include the fundamental mode.

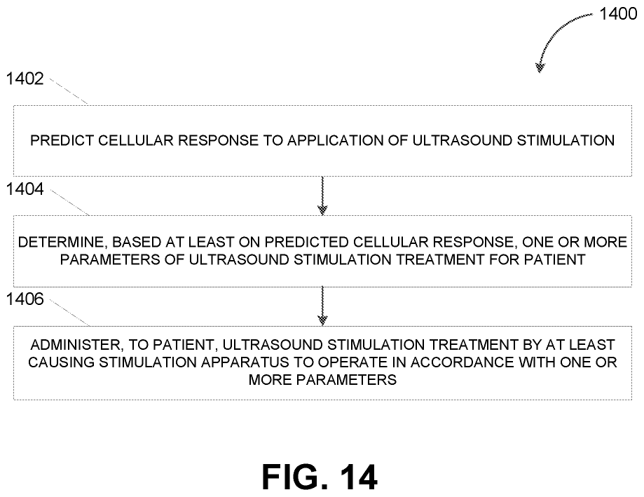

FIG. 14 depicts a flowchart illustrating an example of a process 1400 for ultrasound based cellular stimulation, in accordance with some example embodiments. Referring to FIGS. 1-14, the process 1400 may be performed by the ultrasound-based cellular stimulation system 100, for example, by the stimulation controller 110 and the stimulation apparatus 120.

At 1402, the stimulation controller 110 may predict a cellular response to application of ultrasound stimulation. In some example embodiments, the stimulation controller 110 may apply the deflection model 115 to predict a cellular membrane deflection and the corresponding transmembrane voltage changes induced by the application of an ultrasonic stimulus. As noted, the deflection model 115 may be formulated based on observations made using high-speed digital holographic microscopy (DHM) imaging of cellular membrane deflection (e.g., displacement of cellular membrane) under the influence of various ultrasonic stimulus. For instance, FIG. 11 shows that the measurements of apical cellular membrane deflection due to ultrasound includes a 25-millisecond baseline recording, followed by a 50-millisecond ultrasound stimulus, and a 25-millisecond post-stimulus dwell (see, e.g., FIG. 11(b)), leading to a median deflection of 214 nanometers for human embryonic kidney (HEK293) cells and 159 nanometers for neurons, with a range of 100 to 550 nanometers m across the two tested cell types (see, e.g., FIG. 11(c)). In accordance with the resulting deflection model 115, when subjected to an ultrasonic stimulus in the form of a sinusoidal burst (e.g., a short-term continuously oscillating ultrasound signal of constant amplitude and frequency), maximum membrane deflection is achieved when the ultrasound stimulus is first applied, followed by decay due to viscous losses to the host medium. Moreover, in accordance with the deflection model 115, the magnitude of cellular membrane deflection depends on the stimulation frequency and peak pressure, with lower frequencies and higher pressures producing greater membrane deflection. In some cases, the deflection model 115 applied to predict cellular responses for a patient may be generated using at least some patient-specific data. Alternatively and/or additionally, the deflection model 115 may be generated using at least some non-patient specific data collected from other patients and/or experimental cohorts.

At 1404, the stimulation controller 110 may determine, based at least on the predicted cellular response, one or more parameters of an ultrasound stimulation treatment for a patient. For instance, in some example embodiments, the predicted cellular response may include a magnitude of cellular membrane deflection and/or transmembrane voltage changes induced by the application of an ultrasonic stimulus. Accordingly, the stimulation controller 110 may therefore determine one or more parameters of an ultrasound stimulation treatment for achieving a desired or suitable magnitude of cellular membrane deflection and/or transmembrane voltage changes for a particular patient. For example, the stimulation controller 110 may determine a magnitude and/or a duration of the ultrasonic stimulus for achieving the desired magnitude of cellular membrane deflection and/or transmembrane voltage changes for the patient. In some cases, the stimulation controller 110 may determine, based at least on the cellular responses predicted by the application of the deflection model 115, one or more of an amplitude, a frequency, and/or a pressure (e.g., peak pressure) of the ultrasonic stimulus to achieved the desired magnitude of cellular membrane deflection and/or transmembrane voltage changes for the patient.

At 1406, the stimulation controller 110 may administer, to the patient, the ultrasound stimulation treatment by at least causing the stimulation apparatus 120 to operate in accordance with the one or more parameters. In some example embodiments, the stimulation controller 110 may operate, based at least on the one or more parameters determined in operation 1404, the stimulation apparatus 120 to administer the ultrasound stimulation treatment to the patient. As noted, the stimulation apparatus 120 may include the transducer element 204 formed from a single crystal piezoelectric material (e.g., lithium niobate and the like) having a certain fundamental frequency (e.g., such as approximately 7 megahertz) coupled with a minimal backing epoxy 206. As such, the transducer element 204 of the stimulation apparatus 120 may be driven at high powers without significant heating, thus avoiding tissue damage in the patient. In particular, the stimulation apparatus 120 may be driven at a high power to deliver high magnitudes of acoustic pressure relative to its dimensions (e.g., size, weight, and/or the like) and without significant heating. For example, the magnitude of the acoustic pressure relative to the dimension of the stimulation apparatus 120 may be at least 1 MPa acoustic pressure per gram of the stimulation apparatus 120. In some cases, the stimulation apparatus 120 may include the diffuser 600, which may be disposed on the surface of the transducer element 204 to maximize the uniformity of the ultrasound field created by the stimulation apparatus 120. The diffuser 600 may provide near lossless reduction in the presence of extremely high and low ultrasound intensity, and thus eliminates adverse effects such as heating and tissue damage. Various examples of the stimulation apparatus 120 disclosed herein are also lightweight, portable, and easily secured (e.g., via the magnets 212) to a treatment location of the patient to provide non-invasive or minimally invasive ultrasound based cellular stimulation. In the case of a sonogenetic treatment, for example, the ultrasound based stimulation delivered by the stimulation apparatus 120 may be applied to an area that have been pretreated (e.g., injected) with genetically engineered cells to be more responsive to the ultrasound stimulus.

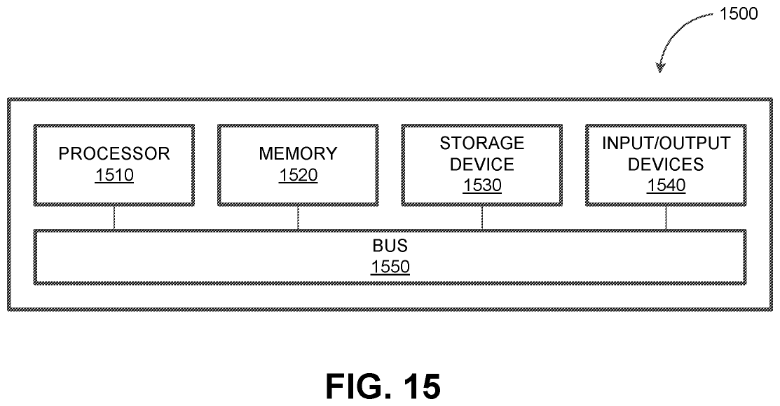

FIG. 15 depicts a block diagram illustrating an example of a computing system 1500 consistent with implementations of the current subject matter. Referring to FIGS. 1-15, the computing system 1500 may implement the stimulation controller 110 and/or any components therein.

As shown in FIG. 15, the computing system 1500 can include a processor 1510, a memory 1520, a storage device 1530, and input/output device 1540. The processor 1510, the memory 1520, the storage device 1530, and the input/output device 1540 can be interconnected via a system bus 550. The processor 1510 is capable of processing instructions for execution within the computing system 1500. Such executed instructions can implement one or more components of, for example, the stimulation controller 110. In some implementations of the current subject matter, the processor 1510 can be a single-threaded processor. Alternately, the processor 1510 can be a multi-threaded processor. The processor 1510 is capable of processing instructions stored in the memory 1520 and/or on the storage device 1530 to display graphical information for a user interface provided via the input/output device 1540.

The memory 1520 is a computer readable medium such as volatile or non-volatile that stores information within the computing system 1500. The memory 1520 can store data structures representing configuration object databases, for example. The storage device 1530 is capable of providing persistent storage for the computing system 1500. The storage device 1530 can be a floppy disk device, a hard disk device, an optical disk device, or a tape device, or other suitable persistent storage means. The input/output device 1540 provides input/output operations for the computing system 1500. In some implementations of the current subject matter, the input/output device 1540 includes a keyboard and/or pointing device. In various implementations, the input/output device 1540 includes a display unit for displaying graphical user interfaces.

According to some implementations of the current subject matter, the input/output device 1540 can provide input/output operations for a network device. For example, the input/output device 1540 can include Ethernet ports or other networking ports to communicate with one or more wired and/or wireless networks (e.g., a local area network (LAN), a wide area network (WAN), the Internet).

In some implementations of the current subject matter, the computing system 1500 can be used to execute various interactive computer software applications that can be used for organization, analysis and/or storage of data in various (e.g., tabular) format (e.g., Microsoft Excel®, and/or any other type of software). Alternatively, the computing system 1500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1540. The user interface can be generated and presented to a user by the computing system 1500 (e.g., on a computer screen monitor, etc.).

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
a stimulation apparatus; and
a stimulation controller, comprising:
at least one data processor; and
at least one memory storing instructions which, when executed by the at least one data processor, cause operations comprising:
predicting a cellular response to an application of ultrasound stimulation;
determining, based at least on the predicted cellular response, one or more parameters of an ultrasound stimulation treatment for a patient; and
administering, to the patient, the ultrasound stimulation treatment by at least causing the stimulation apparatus to operate in accordance with the one or more parameters.

2. The system of claim 1, wherein the stimulation apparatus includes a transducer element formed from a crystal piezoelectric material.

3. The system of claim 2, wherein the crystal piezoelectric material comprises one or more of lithium niobate, lithium tantalate, quartz, and lithium tetraborate.

4. The system of claim 2, wherein a diffuser is disposed on a first surface of the transducer element to reduce an intensity difference in an ultrasound stimulus generated by the transducer element.

5. The system of claim 4, wherein the diffuser comprises one or more wells machined in a substrate to form a plurality of pillars having varying height.

6. The system of claim 5, wherein the plurality of pillars are submillimeter in height.

7. The system of claim 4, wherein an epoxy backing is disposed on a second surface of the transducer element.

8. The system of claim 2, wherein the stimulation apparatus includes a connector configured to provide an electrical connection between the transducer element and a power source.

9. The system of claim 8, wherein the connector comprises a micro-miniature coaxial (MMCX) connector with rotary coaxial connections.

10. The system of claim 8, wherein the stimulation apparatus further includes a bracket and a mounting plate configured to house the connector and the transducer element.

11. The system of claim 10, wherein the bracket and the mounting plate further house one or more magnets for securing the stimulation apparatus to a treatment area on the patient.

12. The system of claim 1, wherein the stimulation apparatus is configured to deliver an acoustic pressure in response to a sinusoidal power input.

13. The system of claim 12, wherein a magnitude of the acoustic pressure relative to a dimension of the stimulation apparatus is at least 1 MPa acoustic pressure per gram of the stimulation apparatus.

14. The system of claim 12, wherein the acoustic pressure is in a range between 0.4 MPa to 0.6 MPa when the sinusoidal power input is in a range of 0.5 watts to 2 watts.

15. The system of claim 1, wherein the ultrasound stimulation treatment includes the stimulation apparatus delivering an ultrasonic stimulus to induce, in the patient, a cellular membrane deflection that causes a change in transmembrane voltage in multiple cell types.

16. The system of claim 15, wherein the one or more parameters include a magnitude and/or a duration of the ultrasonic stimulus for achieving a desired magnitude of cellular membrane deflection and/or transmembrane voltage changes in the patient.

17. The system of claim 15, wherein the one or more parameters include an amplitude, a frequency, and/or a peak pressure of the ultrasonic stimulus for achieving a desired magnitude of cellular membrane deflection and/or transmembrane voltage changes in the patient.

18. The system of claim 1, wherein the one or more parameters are determined by applying a deflection model modelling the cellular response to the application of ultrasound stimulation.

19. The system of claim 18, wherein the deflection model is generated based observations of cellular membrane deflection made using high-speed digital holographic microscopy (DHM) imaging.

20. The system of claim 18, wherein the deflection model further models a change in transmembrane voltage based on a change in a capacitance of cellular membrane that corresponds to a change in an area of cellular membrane associated with the cellular membrane deflection.

21-45. (canceled)
